SEQUENCE LISTING (200 sequences)

SEQ ID NO: 1 - RNA, 22 nt, Homo sapiens
  source 1..22: mol_type = transcribed RNA
ttcagatcccagcggtgcctct

SEQ ID NO: 2 - RNA, 21 nt, Homo sapiens
  source 1..21: mol_type = transcribed RNA
gtgggcgggggcaggtgtgtg

SEQ ID NO: 3 - RNA, 22 nt, Homo sapiens
  source 1..22: mol_type = transcribed RNA
acctggcagcagggagcgtcgt

SEQ ID NO: 4 - RNA, 22 nt, Homo sapiens
  source 1..22: mol_type = transcribed RNA
aggcggggcgccgcgggaccgc

SEQ ID NO: 5 - RNA, 22 nt, Homo sapiens
  source 1..22: mol_type = transcribed RNA
aaaagctgggttgagagggcga

SEQ ID NO: 6 - RNA, 20 nt, Homo sapiens
  source 1..20: mol_type = transcribed RNA
gccggggctttgggtgaggg

SEQ ID NO: 7 - RNA, 24 nt, Homo sapiens
  source 1..24: mol_type = transcribed RNA
gatgcgccgcccactgccccgcgc

SEQ ID NO: 8 - RNA, 22 nt, Homo sapiens
  source 1..22: mol_type = transcribed RNA
ctcctggggcccgcactctcgc

SEQ ID NO: 9 - RNA, 20 nt, Homo sapiens
  source 1..20: mol_type = transcribed RNA
ctaggtggggggcttgaagc

SEQ ID NO: 10 - RNA, 22 nt, Homo sapiens
  source 1..22: mol_type = transcribed RNA
aaaagctgggttgagagggcaa

SEQ ID NO: 11 - RNA, 20 nt, Homo sapiens
  source 1..20: mol_type = transcribed RNA
gtgggttggggcgggctctg

SEQ ID NO: 12 - RNA, 22 nt, Homo sapiens
  source 1..22: mol_type = transcribed RNA
ctcggcgcggggcgcgggctcc

SEQ ID NO: 13 - RNA, 17 nt, Homo sapiens
  source 1..17: mol_type = transcribed RNA
ccccgccaccgccttgg

SEQ ID NO: 14 - RNA, 19 nt, Homo sapiens
  source 1..19: mol_type = transcribed RNA
tggatttttggatcaggga

SEQ ID NO: 15 - RNA, 23 nt, Homo sapiens
  source 1..23: mol_type = transcribed RNA
ccccggtgttggggcgcgtctgc

SEQ ID NO: 16 - RNA, 19 nt, Homo sapiens
  source 1..19: mol_type = transcribed RNA
atcccaccactgccaccat

SEQ ID NO: 17 - RNA, 25 nt, Homo sapiens
  source 1..25: mol_type = transcribed RNA
gtgaggcggggccaggagggtgtgt

SEQ ID NO: 18 - RNA, 23 nt, Homo sapiens
  source 1..23: mol_type = transcribed RNA
gtgagtgggagccccagtgtgtg

SEQ ID NO: 19 - RNA, 22 nt, Homo sapiens
  source 1..22: mol_type = transcribed RNA
actgcagtgaaggcacttgtag

SEQ ID NO: 20 - RNA, 22 nt, Homo sapiens
  source 1..22: mol_type = transcribed RNA
ggggctgtgattgaccagcagg

SEQ ID NO: 21 - RNA, 20 nt, Homo sapiens
  source 1..20: mol_type = transcribed RNA
ggatccgagtcacggcacca

SEQ ID NO: 22 - RNA, 22 nt, Homo sapiens
  source 1..22: mol_type = transcribed RNA
agacacatttggagagggaccc

SEQ ID NO: 23 - RNA, 17 nt, Homo sapiens
  source 1..17: mol_type = transcribed RNA
ccccggggagcccggcg

SEQ ID NO: 24 - RNA, 25 nt, Homo sapiens
  source 1..25: mol_type = transcribed RNA
agcggggaggaagtgggcgctgctt

SEQ ID NO: 25 - RNA, 21 nt, Homo sapiens
  source 1..21: mol_type = transcribed RNA
cgggccggaggtcaagggcgt

SEQ ID NO: 26 - RNA, 21 nt, Homo sapiens
  source 1..21: mol_type = transcribed RNA
gtaggtgacagtcaggggcgg

SEQ ID NO: 27 - RNA, 22 nt, Homo sapiens
  source 1..22: mol_type = transcribed RNA
gagggttgggtggaggctctcc

SEQ ID NO: 28 - RNA, 18 nt, Homo sapiens
  source 1..18: mol_type = transcribed RNA
cgggcgtggtggtggggg

SEQ ID NO: 29 - RNA, 23 nt, Homo sapiens
  source 1..23: mol_type = transcribed RNA
ctggcggagcccattccatgcca

SEQ ID NO: 30 - RNA, 22 nt, Homo sapiens
  source 1..22: mol_type = transcribed RNA
actcggcgtggcgtcggtcgtg

SEQ ID NO: 31 - RNA, 22 nt, Homo sapiens
  source 1..22: mol_type = transcribed RNA
agtgggaggccagggcacggca

SEQ ID NO: 32 - RNA, 21 nt, Homo sapiens
  source 1..21: mol_type = transcribed RNA
accactgcactccagcctgag

SEQ ID NO: 33 - RNA, 18 nt, Homo sapiens
  source 1..18: mol_type = transcribed RNA
aaagctgggttgagaagg

SEQ ID NO: 34 - RNA, 23 nt, Homo sapiens
  source 1..23: mol_type = transcribed RNA
gaacgcctgttcttgccaggtgg

SEQ ID NO: 35 - RNA, 21 nt, Homo sapiens
  source 1..21: mol_type = transcribed RNA
tcgaggagctcacagtctagt

SEQ ID NO: 36 - RNA, 17 nt, Homo sapiens
  source 1..17: mol_type = transcribed RNA
accccactcctggtacc

SEQ ID NO: 37 - RNA, 22 nt, Homo sapiens
  source 1..22: mol_type = transcribed RNA
actcaaaatgggggcgctttcc

SEQ ID NO: 38 - RNA, 22 nt, Homo sapiens
  source 1..22: mol_type = transcribed RNA
actgggtaggtggggctccagg

SEQ ID NO: 39 - RNA, 18 nt, Homo sapiens
  source 1..18: mol_type = transcribed RNA
ggtgggcttcccggaggg

SEQ ID NO: 40 - RNA, 24 nt, Homo sapiens
  source 1..24: mol_type = transcribed RNA
agggctggactcagcggcggagct

SEQ ID NO: 41 - RNA, 22 nt, Homo sapiens
  source 1..22: mol_type = transcribed RNA
cagccctcctcccgcacccaaa

SEQ ID NO: 42 - RNA, 23 nt, Homo sapiens
  source 1..23: mol_type = transcribed RNA
caacggaatcccaaaagcagctg

SEQ ID NO: 43 - RNA, 19 nt, Homo sapiens
  source 1..19: mol_type = transcribed RNA
aatggatttttggagcagg

SEQ ID NO: 44 - RNA, 22 nt, Homo sapiens
  source 1..22: mol_type = transcribed RNA
agggggcgcagtcactgacgtg

SEQ ID NO: 45 - RNA, 21 nt, Homo sapiens
  source 1..21: mol_type = transcribed RNA
gcagggacagcaaaggggtgc

SEQ ID NO: 46 - RNA, 18 nt, Homo sapiens
  source 1..18: mol_type = transcribed RNA
ttcccagccaacgcacca

SEQ ID NO: 47 - RNA, 21 nt, Homo sapiens
  source 1..21: mol_type = transcribed RNA
agtggcaaagtctttccatat

SEQ ID NO: 48 - RNA, 22 nt, Homo sapiens
  source 1..22: mol_type = transcribed RNA
aggactggactcccggcagccc

SEQ ID NO: 49 - RNA, 21 nt, Homo sapiens
  source 1..21: mol_type = transcribed RNA
atccagttctctgagggggct

SEQ ID NO: 50 - RNA, 23 nt, Homo sapiens
  source 1..23: mol_type = transcribed RNA
tagcaccatttgaaatcagtgtt

SEQ ID NO: 51 - RNA, 24 nt, Homo sapiens
  source 1..24: mol_type = transcribed RNA
tgaggggcctcagaccgagctttt

SEQ ID NO: 52 - RNA, 22 nt, Homo sapiens
  source 1..22: mol_type = transcribed RNA
ggtggcccggccgtgcctgagg

SEQ ID NO: 53 - RNA, 21 nt, Homo sapiens
  source 1..21: mol_type = transcribed RNA
ggggggatgtgcatgctggtt

SEQ ID NO: 54 - RNA, 21 nt, Homo sapiens
  source 1..21: mol_type = transcribed RNA
atacatgtcagattgtatgcc

SEQ ID NO: 55 - RNA, 20 nt, Homo sapiens
  source 1..20: mol_type = transcribed RNA
actcaaactgtgggggcact

SEQ ID NO: 56 - RNA, 22 nt, Homo sapiens
  source 1..22: mol_type = transcribed RNA
agggccgaagggtggaagctgc

SEQ ID NO: 57 - RNA, 22 nt, Homo sapiens
  source 1..22: mol_type = transcribed RNA
aaaccgttaccattactgagtt

SEQ ID NO: 58 - RNA, 19 nt, Homo sapiens
  source 1..19: mol_type = transcribed RNA
aaaagctgggttgagagga

SEQ ID NO: 59 - RNA, 22 nt, Homo sapiens
  source 1..22: mol_type = transcribed RNA
tagggggcggcttgtggagtgt

SEQ ID NO: 60 - RNA, 18 nt, Homo sapiens
  source 1..18: mol_type = transcribed RNA
cccagcaggacgggagcg

SEQ ID NO: 61 - RNA, 20 nt, Homo sapiens
  source 1..20: mol_type = transcribed RNA
cgggcgtggtggtgggggtg

SEQ ID NO: 62 - RNA, 21 nt, Homo sapiens
  source 1..21: mol_type = transcribed RNA
agggccccccctcaatcctgt

SEQ ID NO: 63 - RNA, 20 nt, Homo sapiens
  source 1..20: mol_type = transcribed RNA
atatgggtttactagttggt

SEQ ID NO: 64 - RNA, 22 nt, Homo sapiens
  source 1..22: mol_type = transcribed RNA
tgcgacattggaagtagtatca

SEQ ID NO: 65 - RNA, 22 nt, Homo sapiens
  source 1..22: mol_type = transcribed RNA
tggcgggggtagagctggctgc

SEQ ID NO: 66 - RNA, 22 nt, Homo sapiens
  source 1..22: mol_type = transcribed RNA
atcaaggatcttaaactttgcc

SEQ ID NO: 67 - RNA, 22 nt, Homo sapiens
  source 1..22: mol_type = transcribed RNA
tgcaagacggatactgtcatct

SEQ ID NO: 68 - RNA, 23 nt, Homo sapiens
  source 1..23: mol_type = transcribed RNA
ccagggggatgggcgagcttggg

SEQ ID NO: 69 - RNA, 22 nt, Homo sapiens
  source 1..22: mol_type = transcribed RNA
tgcggggctagggctaacagca

SEQ ID NO: 70 - RNA, 20 nt, Homo sapiens
  source 1..20: mol_type = transcribed RNA
aaaagtgattgcagtgtttg

SEQ ID NO: 71 - RNA, 24 nt, Homo sapiens
  source 1..24: mol_type = transcribed RNA
gtaggggcgtcccgggcgcgcggg

SEQ ID NO: 72 - RNA, 19 nt, Homo sapiens
  source 1..19: mol_type = transcribed RNA
ggctggtcagatgggagtg

SEQ ID NO: 73 - RNA, 22 nt, Homo sapiens
  source 1..22: mol_type = transcribed RNA
aggactgatcctctcgggcagg

SEQ ID NO: 74 - RNA, 20 nt, Homo sapiens
  source 1..20: mol_type = transcribed RNA
aaaagctgggttgagagggt

SEQ ID NO: 75 - RNA, 19 nt, Homo sapiens
  source 1..19: mol_type = transcribed RNA
gagccagttggacaggagc

SEQ ID NO: 76 - RNA, 21 nt, Homo sapiens
  source 1..21: mol_type = transcribed RNA
agctgtacctgaaaccaagca

SEQ ID NO: 77 - RNA, 22 nt, Homo sapiens
  source 1..22: mol_type = transcribed RNA
aactagtaatgttggattaggg

SEQ ID NO: 78 - RNA, 18 nt, Homo sapiens
  source 1..18: mol_type = transcribed RNA
gggtgagggcaggtggtt

SEQ ID NO: 79 - RNA, 22 nt, Homo sapiens
  source 1..22: mol_type = transcribed RNA
atgggtgaatttgtagaaggat

SEQ ID NO: 80 - RNA, 22 nt, Homo sapiens
  source 1..22: mol_type = transcribed RNA
ggggaactgtagatgaaaaggc

SEQ ID NO: 81 - RNA, 22 nt, Homo sapiens
  source 1..22: mol_type = transcribed RNA
aaggtattgttcagacttatga

SEQ ID NO: 82 - RNA, 21 nt, Homo sapiens
  source 1..21: mol_type = transcribed RNA
gcggtggggccggaggggcgt

SEQ ID NO: 83 - RNA, 23 nt, Homo sapiens
  source 1..23: mol_type = transcribed RNA
gggggccgatacactgtacgaga

SEQ ID NO: 84 - RNA, 22 nt, Homo sapiens
  source 1..22: mol_type = transcribed RNA
tcaatcacttggtaattgctgt

SEQ ID NO: 85 - RNA, 22 nt, Homo sapiens
  source 1..22: mol_type = transcribed RNA
tatacctcagttttatcaggtg

SEQ ID NO: 86 - RNA, 21 nt, Homo sapiens
  source 1..21: mol_type = transcribed RNA
tgggagctaagctatgggtat

SEQ ID NO: 87 - RNA, 22 nt, Homo sapiens
  source 1..22: mol_type = transcribed RNA
agaaggggtgaaatttaaacgt

SEQ ID NO: 88 - RNA, 23 nt, Homo sapiens
  source 1..23: mol_type = transcribed RNA
tgagagtggaattcacagtattt

SEQ ID NO: 89 - RNA, 21 nt, Homo sapiens
  source 1..21: mol_type = transcribed RNA
attgacacttctgtgagtaga

SEQ ID NO: 90 - RNA, 20 nt, Homo sapiens
  source 1..20: mol_type = transcribed RNA
aagtcctgcttctgttgcag

SEQ ID NO: 91 - RNA, 22 nt, Homo sapiens
  source 1..22: mol_type = transcribed RNA
taaagaactcttaaaacccaat

SEQ ID NO: 92 - RNA, 22 nt, Homo sapiens
  source 1..22: mol_type = transcribed RNA
atctgtaagagaaagtaaatga

SEQ ID NO: 93 - RNA, 23 nt, Homo sapiens
  source 1..23: mol_type = transcribed RNA
ggatggttgggggcggtcggcgt

SEQ ID NO: 94 - RNA, 22 nt, Homo sapiens
  source 1..22: mol_type = transcribed RNA
tgttgtacttttttttttgttc

SEQ ID NO: 95 - RNA, 18 nt, Homo sapiens
  source 1..18: mol_type = transcribed RNA
tgagatgacactgtagct

SEQ ID NO: 96 - RNA, 23 nt, Homo sapiens
  source 1..23: mol_type = transcribed RNA
atgagactcatgtaaaacatctt

SEQ ID NO: 97 - RNA, 21 nt, Homo sapiens
  source 1..21: mol_type = transcribed RNA
agaagtggctaataatattga

SEQ ID NO: 98 - RNA, 22 nt, Homo sapiens
  source 1..22: mol_type = transcribed RNA
agcaaggcggcatctctctgat

SEQ ID NO: 99 - RNA, 22 nt, Homo sapiens
  source 1..22: mol_type = transcribed RNA
gcatgtgatgaagcaaatcagt

SEQ ID NO: 100 - RNA, 22 nt, Homo sapiens
  source 1..22: mol_type = transcribed RNA
tggcccaacctattcagttagt

SEQ ID NO: 101 - RNA, 22 nt, Homo sapiens
  source 1..22: mol_type = transcribed RNA
aagctttttgctcgaattatgt

SEQ ID NO: 102 - RNA, 21 nt, Homo sapiens
  source 1..21: mol_type = transcribed RNA
aggggtgctatctgtgattga

SEQ ID NO: 103 - RNA, 21 nt, Homo sapiens
  source 1..21: mol_type = transcribed RNA
tgtggtagatatatgcacgat

SEQ ID NO: 104 - RNA, 22 nt, Homo sapiens
  source 1..22: mol_type = transcribed RNA
aaaagtaatcactgtttttgcc

SEQ ID NO: 105 - RNA, 20 nt, Homo sapiens
  source 1..20: mol_type = transcribed RNA
agacactatacgagtcatat

SEQ ID NO: 106 - RNA, 21 nt, Homo sapiens
  source 1..21: mol_type = transcribed RNA
aaaagtaattgcggtttttgc

SEQ ID NO: 107 - RNA, 21 nt, Homo sapiens
  source 1..21: mol_type = transcribed RNA
aaggtttggatagatgcaata

SEQ ID NO: 108 - RNA, 24 nt, Homo sapiens
  source 1..24: mol_type = transcribed RNA
gctggtttcatatggtggtttaga

SEQ ID NO: 109 - RNA, 22 nt, Homo sapiens
  source 1..22: mol_type = transcribed RNA
tctggtaagagatttgggcata

SEQ ID NO: 110 - RNA, 18 nt, Homo sapiens
  source 1..18: mol_type = transcribed RNA
aaaagtacttgcggattt

SEQ ID NO: 111 - RNA, 23 nt, Homo sapiens
  source 1..23: mol_type = transcribed RNA
tgagcaccacacaggccgggcgc

SEQ ID NO: 112 - RNA, 22 nt, Homo sapiens
  source 1..22: mol_type = transcribed RNA
tttaagaaaacaccatggagat

SEQ ID NO: 113 - RNA, 25 nt, Homo sapiens
  source 1..25: mol_type = transcribed RNA
aggggggcactgcgcaagcaaagcc

SEQ ID NO: 114 - RNA, 21 nt, Homo sapiens
  source 1..21: mol_type = transcribed RNA
tggatgtggaaggagttatct

SEQ ID NO: 115 - RNA, 21 nt, Homo sapiens
  source 1..21: mol_type = transcribed RNA
tcacctggctggcccgcccag

SEQ ID NO: 116 - RNA, 23 nt, Homo sapiens
  source 1..23: mol_type = transcribed RNA
gtgagtgggagccggtggggctg

SEQ ID NO: 117 - RNA, 22 nt, Homo sapiens
  source 1..22: mol_type = transcribed RNA
agaaggcactatgagatttaga

SEQ ID NO: 118 - RNA, 22 nt, Homo sapiens
  source 1..22: mol_type = transcribed RNA
agcagaggcagagaggctcagg

SEQ ID NO: 119 - RNA, 21 nt, Homo sapiens
  source 1..21: mol_type = transcribed RNA
taacgcataatatggacatgt

SEQ ID NO: 120 - RNA, 21 nt, Homo sapiens
  source 1..21: mol_type = transcribed RNA
ggtggatattccttctatgtt

SEQ ID NO: 121 - RNA, 22 nt, Homo sapiens
  source 1..22: mol_type = transcribed RNA
tgcaacttacctgagtcattga

SEQ ID NO: 122 - RNA, 22 nt, Homo sapiens
  source 1..22: mol_type = transcribed RNA
tgagcccctgtgccgcccccag

SEQ ID NO: 123 - RNA, 22 nt, Homo sapiens
  source 1..22: mol_type = transcribed RNA
agctgtaattagtcagttttct

SEQ ID NO: 124 - RNA, 20 nt, Homo sapiens
  source 1..20: mol_type = transcribed RNA
atggagatagatatagaaat

SEQ ID NO: 125 - RNA, 22 nt, Homo sapiens
  source 1..22: mol_type = transcribed RNA
agggacgggacgcggtgcagtg

SEQ ID NO: 126 - RNA, 23 nt, Homo sapiens
  source 1..23: mol_type = transcribed RNA
taaatttcacctttctgagaaga

SEQ ID NO: 127 - RNA, 23 nt, Homo sapiens
  source 1..23: mol_type = transcribed RNA
tagcaatacagtacaaatatagt

SEQ ID NO: 128 - RNA, 22 nt, Homo sapiens
  source 1..22: mol_type = transcribed RNA
ttagcttaaggagtaccagatc

SEQ ID NO: 129 - RNA, 23 nt, Homo sapiens
  source 1..23: mol_type = transcribed RNA
tgtgactgcattatgaaaattct

SEQ ID NO: 130 - RNA, 22 nt, Homo sapiens
  source 1..22: mol_type = transcribed RNA
tctggtatgtagtaggtaataa

SEQ ID NO: 131 - RNA, 21 nt, Homo sapiens
  source 1..21: mol_type = transcribed RNA
tgagggacccaggacaggaga

SEQ ID NO: 132 - RNA, 21 nt, Homo sapiens
  source 1..21: mol_type = transcribed RNA
ttagtgaaggctattttaatt

SEQ ID NO: 133 - RNA, 22 nt, Homo sapiens
  source 1..22: mol_type = transcribed RNA
atcatacaaggacaatttcttt

SEQ ID NO: 134 - RNA, 22 nt, Homo sapiens
  source 1..22: mol_type = transcribed RNA
acaggtgaggttcttgggagcc

SEQ ID NO: 135 - RNA, 21 nt, Homo sapiens
  source 1..21: mol_type = transcribed RNA
agccaagtggaagttacttta

SEQ ID NO: 136 - RNA, 22 nt, Homo sapiens
  source 1..22: mol_type = transcribed RNA
atgctgacatatttactagagg

SEQ ID NO: 137 - RNA, 22 nt, Homo sapiens
  source 1..22: mol_type = transcribed RNA
tcgggcctggggttgggggagc

SEQ ID NO: 138 - RNA, 18 nt, Homo sapiens
  source 1..18: mol_type = transcribed RNA
atcctagtcacggcacca

SEQ ID NO: 139 - RNA, 21 nt, Homo sapiens
  source 1..21: mol_type = transcribed RNA
ttgtgaagaaagaaattctta

SEQ ID NO: 140 - RNA, 23 nt, Homo sapiens
  source 1..23: mol_type = transcribed RNA
aggcctctgtgacgtcacggtgt

SEQ ID NO: 141 - RNA, 21 nt, Homo sapiens
  source 1..21: mol_type = transcribed RNA
taccacagggtagaaccacgg

SEQ ID NO: 142 - RNA, 22 nt, Homo sapiens
  source 1..22: mol_type = transcribed RNA
gggtggggatttgttgcattac

SEQ ID NO: 143 - RNA, 22 nt, Homo sapiens
  source 1..22: mol_type = transcribed RNA
aaattcatgttcaatctaaacc

SEQ ID NO: 144 - RNA, 22 nt, Homo sapiens
  source 1..22: mol_type = transcribed RNA
tcacaaatctataatatgcagg

SEQ ID NO: 145 - RNA, 21 nt, Homo sapiens
  source 1..21: mol_type = transcribed RNA
aatgtagagattgatcaaaat

SEQ ID NO: 146 - RNA, 22 nt, Homo sapiens
  source 1..22: mol_type = transcribed RNA
atcaaataaggactagtctgca

SEQ ID NO: 147 - RNA, 22 nt, Homo sapiens
  source 1..22: mol_type = transcribed RNA
attaaggacatttgtgattgat

SEQ ID NO: 148 - RNA, 22 nt, Homo sapiens
  source 1..22: mol_type = transcribed RNA
atgtagggctaaaagccatggg

SEQ ID NO: 149 - RNA, 22 nt, Homo sapiens
  source 1..22: mol_type = transcribed RNA
cgcgcctgcaggaactggtaga

SEQ ID NO: 150 - RNA, 20 nt, Homo sapiens
  source 1..20: mol_type = transcribed RNA
aataatatcacagtaggtgt

SEQ ID NO: 151 - RNA, 21 nt, Homo sapiens
  source 1..21: mol_type = transcribed RNA
agttaggattaggtcgtggaa

SEQ ID NO: 152 - RNA, 21 nt, Homo sapiens
  source 1..21: mol_type = transcribed RNA
tatggtactccttaagctaac

SEQ ID NO: 153 - RNA, 20 nt, Homo sapiens
  source 1..20: mol_type = transcribed RNA
actgtaaacgctttctgatg

SEQ ID NO: 154 - RNA, 22 nt, Homo sapiens
  source 1..22: mol_type = transcribed RNA
attgtagaacctaagattggcc

SEQ ID NO: 155 - RNA, 21 nt, Homo sapiens
  source 1..21: mol_type = transcribed RNA
tcgtgcatatatctaccacat

SEQ ID NO: 156 - RNA, 22 nt, Homo sapiens
  source 1..22: mol_type = transcribed RNA
atacctcatctagaatgctgta

SEQ ID NO: 157 - RNA, 22 nt, Homo sapiens
  source 1..22: mol_type = transcribed RNA
gagcaggcgaggctgggctgaa

SEQ ID NO: 158 - RNA, 18 nt, Homo sapiens
  source 1..18: mol_type = transcribed RNA
tcgaggagctcacagtct

SEQ ID NO: 159 - RNA, 22 nt, Homo sapiens
  source 1..22: mol_type = transcribed RNA
gtagcaccttgcaggataaggt

SEQ ID NO: 160 - RNA, 24 nt, Homo sapiens
  source 1..24: mol_type = transcribed RNA
agagctcacagctgtccttctcta

SEQ ID NO: 161 - RNA, 22 nt, Homo sapiens
  source 1..22: mol_type = transcribed RNA
ttctggatatgaagacaatcaa

SEQ ID NO: 162 - RNA, 21 nt, Homo sapiens
  source 1..21: mol_type = transcribed RNA
tttccataggtgatgagtcac

SEQ ID NO: 163 - RNA, 20 nt, Homo sapiens
  source 1..20: mol_type = transcribed RNA
aaaagctgggctgagaggcg

SEQ ID NO: 164 - RNA, 22 nt, Homo sapiens
  source 1..22: mol_type = transcribed RNA
taaatagagtaggcaaaggaca

SEQ ID NO: 165 - RNA, 24 nt, Homo sapiens
  source 1..24: mol_type = transcribed RNA
tgggcgaggggtgggctctcagag

SEQ ID NO: 166 - RNA, 22 nt, Homo sapiens
  source 1..22: mol_type = transcribed RNA
atatgtatatgtgactgctact

SEQ ID NO: 167 - RNA, 22 nt, Homo sapiens
  source 1..22: mol_type = transcribed RNA
cggatgagcaaagaaagtggtt

SEQ ID NO: 168 - RNA, 23 nt, Homo sapiens
  source 1..23: mol_type = transcribed RNA
gccccggcgcgggcgggttctgg

SEQ ID NO: 169 - RNA, 22 nt, Homo sapiens
  source 1..22: mol_type = transcribed RNA
taaggcacgcggtgaatgccaa

SEQ ID NO: 170 - RNA, 24 nt, Homo sapiens
  source 1..24: mol_type = transcribed RNA
tatatggacttttctgatacaatg

SEQ ID NO: 171 - RNA, 23 nt, Homo sapiens
  source 1..23: mol_type = transcribed RNA
tgcatcaggccagaagacatgag

SEQ ID NO: 172 - RNA, 22 nt, Homo sapiens
  source 1..22: mol_type = transcribed RNA
aagttctgttatacactcaggc

SEQ ID NO: 173 - RNA, 23 nt, Homo sapiens
  source 1..23: mol_type = transcribed RNA
agcagcattgtacagggctatga

SEQ ID NO: 174 - RNA, 22 nt, Homo sapiens
  source 1..22: mol_type = transcribed RNA
caggtagatatttgataggcat

SEQ ID NO: 175 - RNA, 20 nt, Homo sapiens
  source 1..20: mol_type = transcribed RNA
agagatgaagcgggggggcg

SEQ ID NO: 176 - RNA, 22 nt, Homo sapiens
  source 1..22: mol_type = transcribed RNA
caaaaaccatagttacttttgc

SEQ ID NO: 177 - RNA, 22 nt, Homo sapiens
  source 1..22: mol_type = transcribed RNA
agctctgctgctcactggcagt

SEQ ID NO: 178 - RNA, 22 nt, Homo sapiens
  source 1..22: mol_type = transcribed RNA
ttctggataacatgctgaagct

SEQ ID NO: 179 - RNA, 22 nt, Homo sapiens
  source 1..22: mol_type = transcribed RNA
aaaagtaattgcggattttgcc

SEQ ID NO: 180 - RNA, 22 nt, Homo sapiens
  source 1..22: mol_type = transcribed RNA
tgaagtaccagctactcgagag

SEQ ID NO: 181 - RNA, 24 nt, Homo sapiens
  source 1..24: mol_type = transcribed RNA
actggctagggaaaatgattggat

SEQ ID NO: 182 - RNA, 17 nt, Homo sapiens
  source 1..17: mol_type = transcribed RNA
taagtgcttccatgctt

SEQ ID NO: 183 - RNA, 22 nt, Homo sapiens
  source 1..22: mol_type = transcribed RNA
aacccagtgggctatggaaatg

SEQ ID NO: 184 - RNA, 21 nt, Homo sapiens
  source 1..21: mol_type = transcribed RNA
cttagattagaggatattgtt

SEQ ID NO: 185 - RNA, 22 nt, Homo sapiens
  source 1..22: mol_type = transcribed RNA
gatgagctcattgtaatatgag

SEQ ID NO: 186 - RNA, 22 nt, Homo sapiens
  source 1..22: mol_type = transcribed RNA
gagcaatgtaggtagactgttt

SEQ ID NO: 187 - RNA, 23 nt, Homo sapiens
  source 1..23: mol_type = transcribed RNA
atcacattgccagggattaccac

SEQ ID NO: 188 - RNA, 22 nt, Homo sapiens
  source 1..22: mol_type = transcribed RNA
ggggggtgtggagccagggggc

SEQ ID NO: 189 - RNA, 22 nt, Homo sapiens
  source 1..22: mol_type = transcribed RNA
atagtgggaagctggcagattc

SEQ ID NO: 190 - RNA, 22 nt, Homo sapiens
  source 1..22: mol_type = transcribed RNA
gactgacacctctttgggtgaa

SEQ ID NO: 191 - RNA, 22 nt, Homo sapiens
  source 1..22: mol_type = transcribed RNA
gaaaacgacaatgacttttgca

SEQ ID NO: 192 - RNA, 22 nt, Homo sapiens
  source 1..22: mol_type = transcribed RNA
gagcttattcataaaagtgcag

SEQ ID NO: 193 - RNA, 21 nt, Homo sapiens
  source 1..21: mol_type = transcribed RNA
agcagacttgacctacaatta

SEQ ID NO: 194 - RNA, 22 nt, Homo sapiens
  source 1..22: mol_type = transcribed RNA
aaaggaaagtgtatcctaaaag

SEQ ID NO: 195 - RNA, 22 nt, Homo sapiens
  source 1..22: mol_type = transcribed RNA
tcacaaggtattgactggcgta

SEQ ID NO: 196 - RNA, 23 nt, Homo sapiens
  source 1..23: mol_type = transcribed RNA
agggaaaaaaaaaaggatttgtc

SEQ ID NO: 197 - RNA, 22 nt, Homo sapiens
  source 1..22: mol_type = transcribed RNA
acgcccttcccccccttcttca

SEQ ID NO: 198 - RNA, 26 nt, Homo sapiens
  source 1..26: mol_type = transcribed RNA
aagtagttggtttgtatgagatggtt

SEQ ID NO: 199 - RNA, 22 nt, Homo sapiens
  source 1..22: mol_type = transcribed RNA
agtgcctgctatgtgccaggca

SEQ ID NO: 200 - RNA, 23 nt, Homo sapiens
  source 1..23: mol_type = transcribed RNA
tctggcaagtaaaaaactctcat

(19) United States
(12) Patent Application Publication
(10) Pub. No.: US 2026/0260751 A1
(43) Pub. Date: Sep. 3, 2026

(54) METHODS OF DEVELOPING CANCER DIAGNOSTIC MODELS AND USES THEREOF IN DEVELOPING CANCER DETECTION METHODS

(71) Applicant: miRoncol Diagnostics Ltd., Ottawa (CA)

(72) Inventors: Jason Zhang, San Diego, CA (US); Hai Hu, Johnstown, PA (US)

(21) Appl. No.: 19/164,925

(22) PCT Filed: Mar. 12, 2024
(86) PCT No.: PCT/US2024/019606

§ 371 Date: Sep. 12, 2025

Related U.S. Application Data
(60) Provisional application No. 63/451,751, filed on Mar. 13, 2023.
(60) Provisional application No. 63/538,292, filed on Sep. 14, 2023.

(51) Int. Cl. G16H 50/20; G16B 25/10; G16B 40/00

(52) U.S. Cl. CPC G16H 50/20; G16B 25/10; G16B 40/00

(57) ABSTRACT

A cancer diagnostic model development method is provided, which includes constructing a training set and building a diagnostic model thereon. The training set comprises miRNA expression profiles from non-cancer subjects and cancer patients with two or more cancer types, and building the diagnostic model includes calculating a diagnostic index based on a selected miRNA biomarker set obtained according to the rankings of miRNAs in a differential expression analysis of the miRNA expression profiles in the training set. Methods for detecting a cancer of interest by means of such developed diagnostic model are also provided. A 4-miRNA based diagnostic model demonstrates high performances in a validation set, capable of achieving sensitivity of ≥0.98 while maintaining specificity of 0.99 in detecting multiple cancers including lung cancer, gastric cancer, biliary tract cancer, bladder cancer, prostate cancer, and glioma.

### CROSS-REFERENCE TO RELATED APPLICATIONS

The present application claims the benefit of U.S. Provisional Application No. 63/451,751 filed on Mar. 13, 2023 and U.S. Provisional Application No. 63/538,292 filed on Sep. 14, 2023, whose disclosures are hereby incorporated by reference in their entireties.

### REFERENCE TO SEQUENCE LISTING SUBMITTED ELECTRONICALLY

The content of the electronically submitted sequence listing, file name Top200list.xml, size 172,425 bytes, and date of creation Mar. 12, 2024, filed herewith, is incorporated herein by reference in its entirety.

### FIELD OF THE INVENTION

The present invention relates generally to the technical field of disease screening, detection and diagnosis, and more specifically relates to a method, a kit, a system, and a non-transitory storage medium for the detection of one or multiple human cancers.

### BACKGROUND

Cancer is a challenging and largely a lethal disease for humans, and it is well appreciated that the survival rate for cancer patients can be much higher if the cancer can be detected (or diagnosed) early. However, early-stage cancer patients are often asymptomatic and thus as a matter of fact, these cancer patients are often less likely to be diagnosed.

Currently there are only four cancer types, including breast cancer, colorectal cancer, lung cancer, and cervical cancer, that have a recommended screening method. It is estimated that ⅔ of cancer diagnosis and 70% of cancer deaths are not covered by the existing cancer screen guidelines. Therefore, there is an urgent need for developing a low-cost and ideally noninvasive or minimally invasive test that is capable of detecting multiple cancer types early, i.e. multi-cancer early detection (MCED).

MicroRNAs (i.e. miRNAs) are small single-stranded non-coding RNA molecules of an average of 22 nucleotides long that are encoded by their corresponding genes in the human genome. It is estimated that these miRNAs function to negatively regulate gene expression for more than 50% human genes. Abnormal miRNA expression has been implicated in many human cancers. It is of particular note that miRNAs are often abundant and stable in blood samples of cancer patients, which are extracellular circulating molecules released into circulation by tumor cells, therefore miRNAs have the potential to serve as noninvasive biomarkers for cancer screening and diagnosis. Previously by use of public serum miRNA microarray datasets, a 4-miRNA diagnostic model from a training set that includes only lung cancer patients and matched non-cancer controls were developed, which showed ~80-100% sensitivity for 10 cancer types and ~70% sensitivity for sarcoma and ovarian cancer, while maintaining ~99% specificity (Zhang A, et al. 2022).

### SUMMARY

In a first aspect, this present disclosure provides a method of developing a diagnostic model for detecting a cancer of interest (i.e. cancer diagnostic model development method).

The method comprises the steps of: (1) constructing a training set that comprises expression profiles of a plurality of miRNAs obtained from non-cancer subjects and cancer patients; and (2) developing the diagnostic model based on the training set. Herein, the cancer patients are configured such that there are at least two cancer types among the cancer patients, and the step (2) further comprises the substeps of: (a) performing differential expression analysis over the expression profiles of the plurality of miRNAs by means of a first statistical model such that the plurality of miRNAs are ranked by adjusted p values; and (b) building the diagnostic model based on a selected miRNA biomarker set from the plurality of miRNAs. Herein the selected miRNA biomarker set comprises at least one miRNA, each selected such that it has a ranking no greater than a preset cutoff m (m≥1).

Herein the first statistical model can optionally be selected from one of the following models, which include, but are not limited to: linear model for microarray data (limma) model, logistic regression model, linear discriminant analysis (LDA) model, conditional logistic regression model, lasso regression model, ridge regression model, random forest, support vector machine, or probit regression model. According to some embodiments, limma model is used as the first statistical model.

In certain embodiments of the cancer diagnostic model development method, the substep (b) comprises: calculating a diagnostic index based on the expression profile of the selected miRNA biomarker set, wherein the diagnostic index is calculated based on formula:

$$diagnostic index = {\sum }_{i = 1}^{n} {t}_{i} * {miRNA}_{i} ; \qquad ( I )$$

where n is the total number of the at least one miRNA in the selected miRNA biomarker set (n≤m), miRNAᵢ is the expression level of i^{th} miRNA in the selected miRNA biomarker set (0<i≤n), and tᵢ is a weight for the i^{th} miRNA.

Herein according to some embodiments, tᵢ is a constant number, and thus the diagnostic index takes an unweighted approach. According to some other embodiments, the weight tᵢ is based on a second statistical model. Similar to the first statistical model, the second statistical model can also be selected from one of the following models which include, but are not limited to: linear model for microarray data (limma) model, logistic regression model, linear discriminant analysis (LDA) model, conditional logistic regression model, lasso regression model, ridge regression model, random forest, support vector machine, or probit regression model.

Herein optionally, the first statistical model and the second statistical model can be different or can be substantially same. Further according to some embodiments, limma model is used as both the first statistical model and the second statistical model.

According to some embodiments, the at least one miRNA in the selected miRNA biomarker set are respectively top n ranked miRNAs.

In the cancer diagnostic model development method, the step (2) can optionally further comprise a substep of: (c) evaluating performance of the diagnostic model through cross validation in the training set. Herein, the cross validation can be at least 2 folds (e.g. 2, 3, 5, 10, etc.). Further optionally, the substep (c) comprises at least one of: calculating Area Under Curve (AUC) of Receiver Operating Characteristic (ROC) curves, and evaluating the performance of the diagnostic model based thereon; or calculating specificity and sensitivity of the diagnostic model, and evaluating the performance of the diagnostic model based thereon. It is noted that besides AUC and specificity/sensitivity, other parameters such as overall accuracy, positive likelihood ratio, negative likelihood ratio, positive predictive value, negative predictive value, Youden's J, etc., may also be used for evaluating the performance of the diagnostic model in the above substep (c) of the step (2).

In certain embodiments, the cancer diagnostic model development method may further comprise a step of: (3) validating the diagnostic model based on a validation set. Herein the validation set comprises expression profiles of the selected miRNA biomarker set obtained from non-cancer subjects and cancer patients with the cancer of interest. Further optionally, the step (3) comprises at least one of: calculating AUC of ROC curves, and evaluating the performance of the diagnostic model based thereon; or calculating specificity and sensitivity of the diagnostic model, and evaluating the performance of the diagnostic model based thereon. It is noted that besides AUC and specificity/sensitivity, other parameters such as overall accuracy, positive likelihood ratio, negative likelihood ratio, positive predictive value, negative predictive value, Youden's J, etc., may also be used for validating the diagnostic model in the above step (3).

A system for developing a diagnostic model for detecting a cancer of interest is further provided, which comprises: a processor; and a non-transitory storage medium containing program instructions for execution by the processor. Herein the program instructions are configured to cause the processor to execute various steps of the cancer diagnostic model development method according to any embodiment as described above in the first aspect.

A non-transitory storage medium is also provided, which is configured to store computer-executable program instructions which, when executed by a processor, cause the processor to execute various steps of the cancer diagnostic model development method according to any embodiment as described above in the first aspect.

In a second aspect, the disclosure further provides a method for detecting a cancer of interest from a subject by means of a diagnostic model. Herein the diagnostic model is developed by the cancer diagnostic model development method according to any embodiment as described above.

According to some embodiments, the cancer detection method comprises the following steps:
(A) determining an expression profile of the selected miRNA biomarker set from a biological sample obtained from the subject;
(B) calculating a diagnostic index of the biological sample based on the expression profile of the selected miRNA biomarker set, wherein the diagnostic index is calculated based on formula:

$$diagnostic index = {\sum }_{i = 1}^{n} {t}_{i} * {miRNA}_{i} ; \qquad ( I )$$

where n is the total number of the at least one miRNA in the selected miRNA biomarker set, miRNAᵢ is the expression level of i^{th} miRNA in the selected miRNA biomarker set (0<i≤n), and tᵢ is a weight for the i^{th} miRNA; and
(C) classifying the subject as having the cancer of interest or not based on the calculated diagnostic index, wherein the subject is classified as having the cancer of interest if the calculated diagnostic index is greater than or equal to a pre-determined threshold or as not having the cancer of interest if otherwise.

Herein optionally, each miRNA in the selected miRNA biomarker set is from the top 200 miRNAs as listed in TABLE 1, and n is smaller than or equal to 200.

According to some embodiments of the cancer detection method, the diagnostic index is calculated using weights from limma model.

Herein, according to some embodiments of the cancer detection method, the at least one miRNA in the selected miRNA biomarker set are respectively top n ranked miRNAs.

According to some embodiments, n is no less than 4 and no more than 200, and the classification is capable of achieving an AUC of more than approximately 0.97 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer.

According to some embodiments, n is no less than 4 and no more than 200, and the classification is capable of achieving a sensitivity of at least approximately 0.75 while maintaining a specificity of no less than approximately 0.99 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer. Further according to some embodiments, the classification is capable of achieving a sensitivity of at least approximately 0.80 while maintaining a specificity of no less than approximately 0.99 for detecting lung cancer, colorectal cancer, esophageal cancer, gastric cancer, liver cancer, ovarian cancer, pancreatic cancer, biliary tract cancer, bladder cancer, glioma, or prostate cancer. Further according to some embodiments, the classification is capable of achieving a sensitivity of at least approximately 0.90 while maintaining a specificity of no less than approximately 0.99 for detecting lung cancer, colorectal cancer, esophageal cancer, gastric cancer, pancreatic cancer, biliary tract cancer, bladder cancer, glioma, or prostate cancer. Further according to some embodiments, the classification is capable of achieving a sensitivity of at least approximately 0.95 while maintaining a specificity of no less than approximately 0.99 for detecting lung cancer, esophageal cancer, gastric cancer, biliary tract cancer, bladder cancer, glioma, or prostate cancer. Further according to some embodiments, the classification is capable of achieving a sensitivity of at least approximately 0.99 while maintaining a specificity of no less than approximately 0.99 for detecting lung cancer, esophageal cancer, gastric cancer, biliary tract cancer, bladder cancer, or prostate cancer.

According to some embodiments of the cancer detection method, the selected miRNA biomarker set consists of hsa-miR-5100, hsa-miR-1228-5p, hsa-miR-8073, and hsa-miR-663a. Further according to some embodiments, the classification is capable of achieving a sensitivity of at least approximately 0.75 while maintaining a specificity of no less than approximately 0.99 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer. Further according to some embodiments, the classification is capable of achieving a sensitivity of at least approximately 0.80 while maintaining a specificity value of approximately 0.99 for detecting lung cancer, colorectal cancer, esophageal cancer, gastric cancer, liver cancer, pancreatic cancer, biliary tract cancer, bladder cancer, glioma, or prostate cancer. Further according to some embodiments, the classification is capable of achieving a sensitivity of at least approximately 0.90 while maintaining a specificity value of approximately 0.99 for detecting lung cancer, colorectal cancer, esophageal cancer, gastric cancer, pancreatic cancer, biliary tract cancer, bladder cancer, glioma, or prostate cancer. Further according to some embodiments, the classification is capable of achieving a sensitivity of at least approximately 0.95 while maintaining a specificity value of approximately 0.99 for detecting lung cancer, gastric cancer, biliary tract cancer, bladder cancer, glioma, or prostate cancer. Further according to some embodiments, the classification is capable of achieving a sensitivity of at least approximately 0.99 while maintaining a specificity value of approximately 0.99 for detecting lung cancer, gastric cancer, biliary tract cancer, or bladder cancer.

According to some embodiments of the cancer detection method, the selected miRNA biomarker set consists of hsa-miR-5100, hsa-miR-1228-5p, hsa-miR-8073, hsa-miR-663a, hsa-miR-320a, and the classification is capable of achieving a sensitivity of at least approximately 0.80 while maintaining a specificity value of approximately 0.99 for detecting lung cancer, colorectal cancer, esophageal cancer, gastric cancer, liver cancer, ovarian cancer, pancreatic cancer, biliary tract cancer, bladder cancer, glioma, or prostate cancer.

According to some embodiments of the cancer detection method, the selected miRNA biomarker set consists of top 10 miRNAs from TABLE 1, and the classification is capable of achieving a sensitivity of at least approximately 0.99 while maintaining a specificity value of approximately 0.99 for detecting gastric cancer, esophageal cancer, biliary tract cancer, or prostate cancer.

According to some embodiments of the cancer detection method, the selected miRNA biomarker set consists of top 15 miRNAs from Table 1, wherein the classification is capable of achieving a sensitivity of at least approximately 0.90 while maintaining a specificity value of approximately 0.99 for detecting lung cancer, esophageal cancer, gastric cancer, liver cancer, pancreatic cancer, biliary tract cancer, bladder cancer, glioma, or prostate cancer.

In any embodiment of the cancer detection method as described above, the expression profile of the selected miRNA biomarker set is obtained by means of at least one of Northern Blotting, microarray analysis, RNA-sequencing, or RNA in-situ hybridization, or a nucleic acid amplification procedure, wherein the nucleic acid amplification procedure comprises at least one of reverse-transcription PCR (RT-PCR), quantitative RT-PCR (qRT-PCR), or digital RT-PCR.

In any embodiment of the cancer detection method as described above, the biological sample is a liquid biopsy sample selected from a group consisting of a blood sample, a serum sample, a plasma sample, a urine sample, a saliva sample, and a sputum sample.

A system for detecting a cancer of interest from a subject is also provided, which comprises: a processor; and a non-transitory storage medium containing program instructions for execution by the processor. Herein the program instructions are configured to cause the processor to execute various steps of the cancer detection method according to any embodiment as described above in the second aspect.

A non-transitory storage medium is also provided, which is configured to store computer-executable program instructions which, when executed by a processor, cause the processor to execute various steps of the cancer detection method according to any embodiment as described above in the second aspect.

Unless defined elsewhere, the terms as used throughout the disclosure are defined as follows.

In general terms, a “subject” means a mammal such as a primate including a human and a chimpanzee, a pet animal including a dog and a cat, a livestock animal including cattle, a horse, sheep, and a goat, and a rodent including a mouse and a rat. The term “healthy subject” also means such a mammal without the cancer to be detected. It is to be noted that the whole disclosure concerns more specifically human subjects, but can optionally be applied to other non-human mammals as well.

Unless indicated or defined otherwise, the terms or abbreviations such as “nucleic acid”, “nucleotide”, “polynucleotide”, “DNA”, “RNA”, and “miRNA” abide by common use in the art.

As used herein, the term “polynucleotide” is interchangeable with “nucleic acid”, and refers to as a nucleic acid including all of RNA, DNA, and RNA/DNA (chimera). The DNA includes all of cDNA, genomic DNA, and synthetic DNA. The RNA includes all of total RNA, mRNA, rRNA, miRNA, siRNA, snoRNA, snRNA, non-coding RNA and synthetic RNA.

As used herein, the term “fragment” is a polynucleotide having a nucleotide sequence having a consecutive portion of a polynucleotide and desirably has a length of 15 or more nucleotides, e.g. 15, 16, 17, 18, 19, etc. nucleotides.

As used herein, the term “gene” is intended to include not only RNA and double-stranded DNA but also each single-stranded DNA such as a plus strand (or a sense strand) or a complementary strand (or an antisense strand) constituting the duplex. The gene is not particularly limited by its length. As used herein, the “gene” includes all of double-stranded DNA including human genomic DNA, single-stranded DNA (plus strand) including cDNA, single-stranded DNA having a sequence complementary to the plus strand (complementary strand), miRNA (miRNA), and their fragments, and their transcripts, unless otherwise specified. The “gene” includes not only a “gene” represented by a particular nucleotide sequence (or SEQ ID NO) but “nucleic acids” encoding RNAs having biological functions equivalent to an RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 200 or a nucleotide sequence derived from the nucleotide sequence by the replacement of the nucleotide “U” (or “u”) with the nucleotide “T” (or “t”). The “gene” is not particularly limited by its functional region and can contain, for example, an expression control region, a coding region, an exon, or an intron. The “gene” may be contained in a cell or may exist alone after being released into the outside of a cell. Alternatively, the “gene” may be in a state enclosed in a vesicle called exosome.

Within the scope of the whole disclosure, the term “microRNA” or “miRNA” is intended to mean a 15- to 25-nucleotide non-coding RNA that is transcribed as an RNA precursor having a hairpin-like structure, cleaved by a dsRNA-cleaving enzyme which has RNase III cleavage activity, integrated into a protein complex called RISC, and involved in the suppression of translation of mRNA, unless otherwise specified. The term “miRNA” used As used herein includes not only a “miRNA” represented by a particular nucleotide sequence (or SEQ ID NO) but a precursor of the “miRNA” (pre-miRNA or pri-miRNA), and miRNAs having biological functions equivalent thereto, for example, a congener (i.e., a homolog or an ortholog), a variant (e.g., a genetic polymorph), and a derivative. Such a precursor, a congener, a variant, or a derivative can be specifically identified using miRBase Release 20 (Kozomara and Griffiths-Jones, 2010), and examples thereof can include an “miRNA” having a nucleotide sequence hybridizing under stringent conditions described later to a complementary sequence of any particular nucleotide sequence represented by any of SEQ ID NOs: 1 to 200. The term “miRNA” as used herein may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

As used herein, the term “probe” includes a polynucleotide that is used for specifically detecting an RNA resulting from the expression of a gene or a polynucleotide derived from the RNA, and/or a polynucleotide complementary thereto.

As used herein, the term “primer”, or “amplification primers” includes a polynucleotide that specifically recognizes and amplifies an RNA resulting from the expression of a gene or a polynucleotide derived from the RNA, and/or a polynucleotide complementary thereto.

In this context, the complementary polynucleotide (complementary strand or reverse strand) means a polynucleotide in a complementary base relationship based on A:T (U) and G:C base pairs with the full-length sequence of a polynucleotide consisting of a nucleotide sequence defined by any of SEQ ID NOs: 1 to 200 or a nucleotide sequence derived from the nucleotide sequence by the replacement of the nucleotide “U” (or “u”) with the nucleotide “T” (or “t”), or a partial sequence thereof (here, this full-length or partial sequence refers to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

As used herein, the term “stringent conditions” refers to conditions under which a nucleic acid probe hybridizes to its target sequence to a larger extent (e.g., a measurement value equal to or larger than a mean of background measurement values+a standard deviation of the background measurement values×2) than that for other sequences. The stringent conditions are dependent on a sequence and differ depending on an environment where hybridization is performed. A target sequence complementary 100% to the nucleic acid probe can be identified by controlling the stringency of hybridization and/or washing conditions. Specific examples of the “stringent conditions” will be mentioned later.

As used herein, the term “Tm value” means a temperature at which the double-stranded moiety of a polynucleotide is denatured into single strands so that the double strands and the single strands exist at a ratio of 1:1.

As used herein, the term “variant” means, in the case of a nucleic acid, a natural variant attributed to polymorphism, mutation, or the like; a variant containing the deletion, substitution, addition, or insertion of 1, 2, or 3 or more nucleotides in a nucleotide sequence represented by any of SEQ ID NOs: 1 to 200 or a nucleotide sequence derived from the nucleotide sequence by the replacement of the nucleotide “U” (or “u”) with the nucleotide “T” (or “t”), or a partial sequence thereof; a variant containing the deletion, substitution, addition, or insertion of 1 or 2 or more nucleotides in a nucleotide sequence of a premature miRNA of a sequence represented by any of SEQ ID NOs: 1 to 200 or a nucleotide sequence derived from the nucleotide sequence by the replacement of the nucleotide “U” (or “u”) with the nucleotide “T” (or “t”), or a partial sequence thereof; a variant that exhibits % identity of approximately 90% or higher, approximately 95% or higher to each of these nucleotide sequences or the partial sequences thereof; or a nucleic acid hybridizing under the stringent conditions defined above to a polynucleotide or an oligonucleotide comprising each of these nucleotide sequences or the partial sequences thereof. A variant can be prepared by use of a well-known technique such as site-directed mutagenesis or PCR-based mutagenesis.

The term “percent (%) identity” can be determined with or without an introduced gap, using a protein or gene search system based on BLAST or FASTA described above (Zhang et al., 2000; Altschul et al. 1990; Pearson et al. 1988).

The term “derivative” is meant to include a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen et al. 1991), and LNA (locked nucleic acid; Obika et al. 1998) without any limitation.

The “nucleic acid” capable of specifically binding to a polynucleotide selected from the miRNAs described above is a synthesized or prepared nucleic acid and specifically includes a “nucleic acid probe” or a “primer”. The “nucleic acid” is utilized directly or indirectly for detecting the presence or absence of cancer in a subject, for diagnosing the severity, the degree of amelioration, or the therapeutic sensitivity of cancer, or for screening for a candidate substance useful in the prevention, amelioration, or treatment of cancer. The “nucleic acid” includes a nucleotide, an oligonucleotide, and a polynucleotide capable of specifically recognizing and binding to a transcript represented by any of SEQ ID NOs: 1 to 200, or a synthetic cDNA nucleic acid thereof in vivo, particularly, in a sample such as a body fluid (e.g., blood or urine), in relation to the development of cancer. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” as used herein is interchangeable with the term “examination”, “measurement”, or “detection or decision support”. As used herein, the term “evaluation” is meant to include diagnosis or evaluation support on the basis of examination results or measurement results.

As used within the scope of the disclosure, each of the terms “p-value”, “accuracy”, “AUC”, “sensitivity”, and “specificity” is generally to be understood to have the common definition that is well appreciated by people skilled in the art, and is specifically defined as follows:

As used herein, the term “p-value”, “p value”, “p-value”, “p value”, or “p” refers to a probability at which a more extreme statistic than that actually calculated from data under a null hypothesis is observed in a statistical test. Thus, smaller “p value” means more significant difference between subjects to be compared.

The term “AUC” means area under the curve of a Receiver Operating Characteristic curve. The term “accuracy” means a value of (the number of true positives+the number of true negatives)/(the total number of cases). The accuracy indicates the ratio of samples that were correctly identified to all samples and serves as a primary index to evaluate detection performance.

As used herein, the term “sensitivity” means a value of (the number of true positives)/(the number of true positives+the number of false negatives). High sensitivity allows cancer to be detected, leading to clinical treatment interventions.

As used herein, the term “specificity” means a value of (the number of true negatives)/(the number of true negatives+the number of false positives). High specificity prevents needless extra examination for healthy subjects misjudged as being cancer patients, leading to reduction in burden on patients and reduction in medical expense.

Unless specified elsewhere, the following summarizes the available technologies that can be used for the determination of the expression profile of the miRNA biomarker set.

It is to be noted that determination of the expression profile of the miRNA biomarker set substantially includes the determination of the expression level of each and every miRNA contained in the miRNA biomarker set. Preferably, expression levels for all of the miRNA contained in the miRNA biomarker set can be determined simultaneously in one single experiment that is well-controlled. Yet optionally, it is possible that expression levels of these miRNAs are determined in more than one experiment and by different experiment procedure.

As used herein, measuring or detecting the expression of any of the miRNAs contained in the miRNA biomarker set comprises measuring or detecting any nucleic acid transcript corresponding to the miRNA.

Typically, expression can be detected or measured on the basis of miRNA or corresponding reverse transcribed cDNA levels. Any quantitative or qualitative method for measuring RNA levels, or cDNA levels can be used. Suitable methods of detecting or measuring miRNA or cDNA levels include, for example, Northern Blotting, microarray analysis, RNA-sequencing, RNA in-situ hybridization, or a nucleic acid amplification procedure, such as reverse-transcription PCR (RT-PCR) or real-time RT-PCR, also known as quantitative RT-PCR (qRT-PCR), or digital RT-PCR. Such methods are well known in the art (see e.g., Sambrook et al. 2012). Other techniques include digital, multiplexed analysis of gene expression, such as the nCounter® (NanoString Technologies, Seattle, WA) gene expression assays, which are further described in US20100112710 and US20100047924.

Detecting a nucleic acid of interest generally involves hybridization between a target (e.g. miRNA or cDNA) and a probe. Sequences of the miRNAs used in various cancer gene expression profiles are known. Therefore, one of skills in the art can readily design hybridization probes for detecting those miRNAs (see e.g., Sambrook et al. 2012). For example, polynucleotide probes that specifically bind to the miRNA transcripts described herein (or cDNA synthesized therefrom) can be created using the nucleic acid sequences of the miRNA or cDNA targets themselves by routine techniques (e.g., PCR or synthesis). As used herein, the term “probe” means a part or portion of a polynucleotide sequence comprising about 10 or more contiguous nucleotides, about 15 or more contiguous nucleotides, about 20 or more contiguous nucleotides. In certain embodiments, the polynucleotide probes will comprise 10 or more nucleic acids, 15 or more nucleic acids, or 20 or more nucleic acids. In order to confer sufficient specificity, the probe may have a sequence identity to a complement of the target sequence of about 90% or more, such as about 95% or more (e.g., about 98% or more or about 99% or more) as determined, for example, using the well-known Basic Local Alignment Search Tool (BLAST) algorithm (available through the National Center for Biotechnology Information (NCBI), Bethesda, MD).

Each probe may be substantially specific for its target, to avoid any cross hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of miRNAs being analyzed, for example hybridization to the polyA tail would not provide specificity. If a target has multiple splice variants, it is possible to design a hybridization reagent that recognizes a region common to each variant and/or to use more than one reagent, each of which may recognize one or more variants.

Stringency of hybridization reactions is readily determinable by one of ordinary skill in the art, and generally is an empirical calculation dependent upon probe length, washing temperature, and salt concentration. In general, longer probes may require higher temperatures for proper annealing, while shorter probes may require lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

“Stringent conditions” or “high stringency conditions,” as defined herein, are identified by, but not limited to, those that: (1) use low ionic strength and high temperature for washing, for example 0.015 M sodium chloride/0.0015 M sodium citrate/0.1% sodium dodecyl sulfate at 50° C.; (2) use during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) use 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash of 0.1×SSC containing EDTA at 55° C. “Moderately stringent conditions” are described by, but not limited to, those in Sambrook et al. 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In certain embodiments, microarray analysis, Northern blot, RNA in-situ hybridization, or a PCR-based method is used. In this respect, measuring the expression of the foregoing miRNAs in a biological sample can comprise, for instance, contacting a sample containing or suspected of containing cancer cells with polynucleotide probes specific to the miRNAs of interest, or with primers designed to amplify a portion of the miRNAs of interest, and detecting binding of the probes to the nucleic acid targets or amplification of the nucleic acids, respectively. Detailed protocols for designing PCR primers are known in the art (see e.g., Sambrook et al. 2012). In certain embodiments, miRNAs obtained from a sample may be subjected to qRT-PCR. Reverse transcription may occur by any methods known in the art, such as through the use of an Omniscript RT Kit (Qiagen). The resultant cDNA may then be amplified by any amplification technique known in the art. miRNA expression may then be analyzed through the use of, for example, control samples as described below. As described herein, the over- or under-expression of miRNAs relative to controls may be measured to determine a miRNA expression profile for an individual biological sample. Similarly, detailed protocols for preparing and using microarrays to analyze miRNA expression are known in the art and described herein.

As used herein, RNA-sequencing (RNA-seq), also called Whole Transcriptome Shotgun Sequencing, refers to any of a variety of high-throughput sequencing techniques used to detect the presence and quantity of RNA transcripts in real time. See Wang, Z., M. Gerstein, and M. Snyder, RNA-Seq: a revolutionary tool for transcriptomics, NAT REV GENET, 2009. 10(1): p. 57-63. RNA-seq can be used to reveal a snapshot of a sample's miRNAs from a genome at a given moment in time. In certain embodiments, miRNA is converted to cDNA fragments via reverse transcription prior to sequencing, and, in certain embodiments, miRNA can be directly sequenced without conversion to cDNA. Adaptors may be attached to the 5′ and/or 3′ ends of the miRNAs, and the miRNA or cDNA may optionally be amplified, for example by PCR. The fragments are then sequenced using high-throughput sequencing technology, such as, for example, those available from Roche (e.g., the 454 platform), Illumina, Inc., and Applied Biosystem (e.g., the SOLiD system).

The following are to be noted.

It is noted that the background description includes information that may be useful in understanding the present invention. However, it is not an admission that any of the information provided herein is prior art or relevant to the presently claimed subject matter, or that any publication specifically or implicitly referenced is prior art.

It is further noted that in interpreting both the specification and the claims, all terms should be interpreted in the broadest possible manner consistent with the context. In particular, the terms “comprise” and “comprising” should be interpreted as referring to elements, components, or steps in a non-exclusive manner, indicating that the referenced elements, components, or steps may be present, or utilized, or combined with other elements, components, or steps that are not expressly referenced. The meaning of “a,” “an,” and “the” includes plural reference unless the context clearly dictates otherwise. Also, as used in the description herein, the meaning of “in” includes “in” and “on” unless the context clearly dictates otherwise. The numbers expressing quantities of ingredients, properties such as concentration, reaction conditions, and so forth, used to describe and claim certain embodiments of the invention are to be understood as being modified in some instances by the term “about” As used herein, the terms “about”, “around”, “approximately” or alike, when referring to a specified, measurable value (such as a parameter, an amount, a temporal duration, and the like), is meant to encompass the specified value and variations of and from the specified value, such as variations of +/−20% or less, alternatively variations of +/−10% or less, . . . and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. The use of any and all examples, or exemplary language (e.g., “such as”) provided with respect to certain embodiments herein is intended merely to better illuminate the invention and does not pose a limitation on the scope of the invention otherwise claimed.

### BRIEF DESCRIPTION OF THE DRAWINGS

FIG. 1 illustrates a block diagram of a cancer diagnostic model development method provided by some embodiments of the disclosure.

FIG. 2 illustrates a computerized system according to some embodiments of the disclosure.

FIGS. 3A-3B together illustrate the flow of datasets and study design, with FIG. 3A showing the construction of the train and validation datasets, with FIG. 3B showing the study design of model development and validation.

FIGS. 4A-4B shows the development of the diagnostic models by cross validation in the training set, with FIG. 4A showing the process of cross validation, and FIG. 4B showing the performances of different diagnostic model with the top N miRNAs (N=1, 2, 3, . . . , 40).

FIG. 5 compares the diagnostic performances of different diagnostic models, which vary by the number N of top miRNAs (N is between 4 and 200).

FIGS. 6A-6B show diagnostic performances of the 4-miRNA model in the multi-cancer Train Set, with FIG. 6B showing the ROC of the 4-miRNA model, and FIG. 6C showing the scatterplot of the diagnostic index.

FIGS. 7A-7D show diagnostic performances of the 4-miRNA model in Validation Set 1 (the lung cancer validation dataset), with FIG. 7A showing the ROC of the 4-miRNA model, FIG. 7B showing the scatterplot of the diagnostic index, FIG. 7C showing the scatterplot of the diagnostic index from pre- vs. post-operation serum samples, and FIG. 7D showing the scatterplot of the diagnostic index in clinical subsets. ADC: adenocarcinoma; SqCC: squamous cell carcinoma; LCC: large cell carcinoma; SCLC: small cell lung cancer.

FIGS. 8A-8B show diagnostic performances of the 4-miRNA model in the Validation Set 2 and 3, with FIG. 8A showing the scatterplot of the diagnostic index in the Validation Set 2, and FIG. 8B showing the scatterplot of the diagnostic index in the Validation Set 3.

### DETAILED DESCRIPTION

It is commonly believed that if one wants to develop a diagnostic model for detecting a particular disease (e.g. a particular cancer type), one shall use data (e.g. clinical data, molecular data, etc.) specifically for that particular disease only and shall avoid using data from a different disease (e.g. a different cancer type), because the data from a different disease (e.g. a different cancer type) would unfavorably bring noise in the development of the diagnostic model, therefore causing the diagnostic model thus developed to perform less ideally.

In an effort to develop a cancer detection method, we used serum miRNA microarray datasets obtained from more than one cancer type. Unexpectedly we found that the inclusion of miRNA expression data from more than one cancer type has led to the development of a diagnostic model that can sensitively and reliably detect a particular one cancer type. Briefly in our study which will be described in greater detail below in the Examples part, our training set used miRNA expression datasets from a total of 7 cancer types (lung cancer, ovarian cancer, liver cancer, bladder cancer, esophageal cancer, gastric cancer, and prostate cancer), and surprisingly, our cross-validation using the training set and a subsequent validation using different validation sets showed that the diagnostic models developed based on the data from the 7 cancer types work unexpectedly very well not only for these 7 cancer types, but surprisingly also for most of the other cancers tested (biliary tract cancer, colorectal cancer, glioma, pancreatic cancer, and sarcoma).

On the basis of this work, a cancer diagnostic model development approach is provided in a first aspect of this disclosure.

This model development approach substantially comprises the development of a diagnostic model for detecting a cancer of interest using data from multiple cancer types.

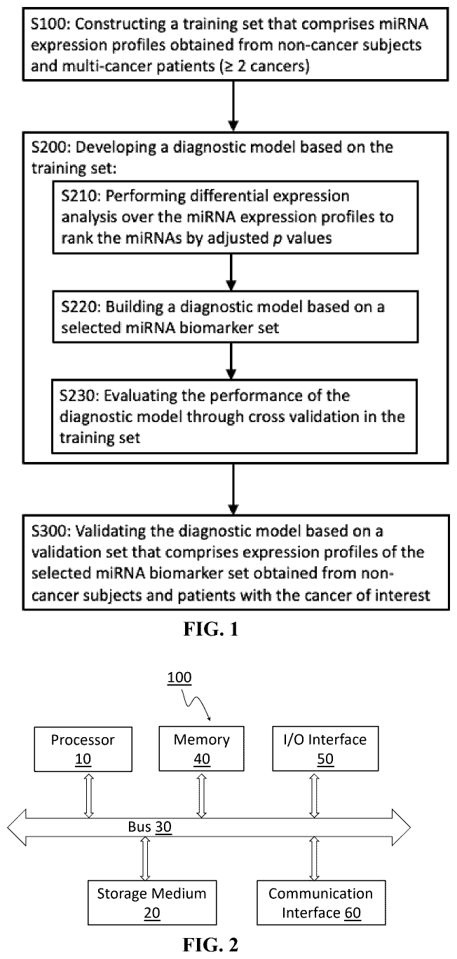

As illustrated in FIG. 1, a method for developing a diagnostic model for detecting a cancer of interest as provided according to some embodiments of the disclosure is provided, which substantially comprises the following three major steps S100-S300:

S100: Constructing a training set that comprises miRNA expression profiles obtained from non-cancer subjects and multi-cancer patients (≥2 cancers);

S200: Developing a diagnostic model based on the training set; and

S300: Validating the diagnostic model based on a validation set that comprises expression profiles of the selected miRNA biomarker set obtained from non-cancer subjects and patients with the cancer of interest.

In the step S100, a training set is constructed which is to be used for building the diagnostic model in the step S200. The training set comprises expression profiles of a plurality of miRNAs (i.e. miRNA expression profiles) that are obtained from non-cancer subjects and multi-cancer patients, which are substantially used as controls and cases respectively when developing the cancer detection diagnostic model in the step S200.

Herein, there are at least two cancer types among the multi-cancer patients. To be more specific, there are a total of N cancer types among the cancer patients (N≥2), i.e., these multi-cancer patients may include a first subset of patients having a first cancer type, a second subset of patients having a second cancer type, . . . , an N^{th} subset of patients having a Nth cancer type. According to some embodiments, the at least two cancer types can include a cancer type that is same as the cancer of interest. According to some other embodiments, the at least two cancer types do not include the cancer of interest.

As used herein, and throughout the whole disclosure as well, the term “cancer” means a specific “cancer type”, defined as a specific type of human disease that involves abnormal increases in the number of cells of a particular cell type (e.g. epithelial cells, connective tissue cells, blood cells, etc.), with the potential to invade or spread to other parts of the body. The cancer types as covered herein may include, but are not limited to, carcinomas, sarcomas, lymphomas, germ cell tumors, blastomas, etc., and may also include both benign tumors and malignant tumors. Non-limiting examples of a cancer type may include lung cancer, breast cancer, esophageal cancer, prostate cancer, gastric cancer, pancreatic cancer, liver cancer, ovarian cancer, colorectal cancer, biliary tract cancer, kidney cancer, bladder cancer, brain tumor (e.g. glioma), sarcoma (e.g. osteosarcoma, chondrosarcoma, fibrosarcoma, etc.), leukemia, lymphoma, germ cell tumor (e.g. germinoma, seminoma, etc.), hepatoblastoma, medulloblastoma, etc.

In one illustrating example where the cancer of interest is pancreatic cancer, the cancer diagnostic model development method as described above can be used to develop a diagnostic model that can be specifically utilized for the detection of pancreatic cancer. As such, the miRNA expression profiles obtained from non-cancer subjects (i.e. control) and multi-cancer patients (i.e. case) can be utilized. Herein optionally the multi-cancer patients may include one subset of pancreatic cancer patient, and at least one other subset of other cancer patients (e.g. lung cancer patients, glioma patients, etc.). Optionally, the multi-cancer patients may include several subsets of cancer patients that are not pancreatic cancer patients (e.g. lung cancer patients, glioma patients, prostate cancer).

As used herein and throughout the disclosure, the “expression profile” refers to a collection of expression information about a given set of biological molecules (e.g. miRNAs, proteins, DNA molecules, etc.), which may include the expression data obtained for each member molecule in the given set of biological molecules. There may be a variety of ways to obtain the expression profile. For example, a miRNA expression profile as described in this disclosure can optionally be obtained by means of Northern Blotting, microarray analysis, RNA-sequencing, or RNA in-situ hybridization, or can optionally be obtained by means of a nucleic acid amplification procedure, comprising reverse-transcription PCR (RT-PCR), quantitative RT-PCR (qRT-PCR), or digital RT-PCR. The miRNA expression profile may be obtained directly from the cancerous or tumor tissues from a patient having a particular cancer type, or may be obtained from some other tissues from the patient, such as blood, serum, plasma, saliva, sweat, etc. therefrom. In this latter case, the miRNAs thus assayed in the expression profile may exist as cell-free miRNAs that are released from tumor cells into the circulation. The miRNA expression profiles obtained regardless of the sample origins or assaying approaches shall be interpreted to be covered in this disclosure.

According to certain embodiments, the step S200 of developing a diagnostic model based on the training set comprises the following sub-steps:
S210: Performing differential expression analysis over the miRNA expression profiles to rank the miRNAs by adjusted p values; and
S220: Building a diagnostic model based on a selected miRNA biomarker set.

Herein, the sub-step S210 of performing differential expression analysis over the miRNA expression profiles to thereby rank the miRNAs by adjusted p values. The differential expression analysis has been carried out widely, for example, in a report by Zhang et al. 2014, whose disclosure is incorporated by reference in its entirety. can be carried out by means of a first statistical model, which may be selected from one of the following statistical models: linear model for microarray data (limma) model, logistic regression model, linear discriminant analysis (LDA) model, conditional logistic regression model, lasso regression model, ridge regression model, random forest, support vector machine, or probit regression model. It is noted that other statistical models can also be used as the first statistical model herein.

Each of the terms “linear models for microarray data (limma) model” (Ritchie et al. 2015), “logistic regression model” (Venable and Ripley 2002), “linear discriminant analysis (LDA) model” (Venable and Ripley 2002), “conditional logistic regression model” (Venable and Ripley 2002), “lasso regression model” (Tibshirani 1996), “ridge regression model” (Hoerl and Kennard 1970), “random forest” (Ripley 1996), “support vector machine” (Ripley 1996), and “probit regression model” (Venable and Ripley 2002) is substantially a probability-modeling statistical model that abides by the definition commonly appreciated by people skilled in the field, the details of which can be referenced by the reference included immediately behind.

As used herein, the term “adjusted p value” means ap value adjusted for multiple testing. The ranking of miRNAs (e.g. 1, 2, 3, . . . , etc.) are based on the adjusted p values such that the miRNA with the smallest adjusted p value has a highest rank of 1, and a miRNA with a smaller adjusted p value has a higher rank.

Herein, in the sub-step S220 of building a diagnostic model based on a selected miRNA biomarker set, the selected miRNA biomarker set substantially comprises at least one miRNA, and each miRNA in the selected miRNA biomarker set is selected such that its ranking is lower than or equal to (i.e. no greater than) a preset cutoff m. Herein, the “predetermined cutoff” can be any integer greater than or equal to one (i.e. m≥1; e.g. m=1, 2, 3, 5, 10, 15, 20, 50, 100, 200, etc.).

According to some embodiments of the cancer diagnostic model development method, the sub-step S220 of building a diagnostic model based on the selected miRNA biomarker set comprises: calculating a diagnostic index based on the expression profile of the selected miRNA biomarker set, wherein the diagnostic index is calculated based on formula:

$$diagnostic index = {\sum }_{i = 1}^{n} {t}_{i} * {miRNA}_{i} ; \qquad ( I )$$

In the above formula (I), n is the total number of the at least one miRNA in the selected miRNA biomarker set, miRNAᵢ is the expression level of i^{th} miRNA in the selected miRNA biomarker set, i is an integer greater than zero and smaller than or equal to n (i.e. 0<i≤n); and tᵢ is a weight for the i^{th} miRNA. Furthermore, the total number of the miRNAs in the selected miRNA biomarker set n is smaller than or equal to the preset cutoff m (i.e. n≤m).

According to some embodiments, an unweighted approach is applied for calculating the diagnostic index, and as such, the weight for each miRNA in the selected miRNA biomarker set is an equal number (e.g. 1, 2, 0.5, etc.). In other word, for any miRNAᵢ in the selected miRNA biomarker set, tᵢ=C (i.e. constant number).

According to some other embodiments, a weighted approach is applied for calculating the diagnostic index, and as such, the weight tᵢ for the i^{th} miRNA is based on a second statistical model.

Herein depending on different embodiments, the second statistical model can be same as, or different from, the first statistical model as mentioned above, and can be selected from limma model, logistic regression model, LDA model, conditional logistic regression model, lasso regression model, ridge regression model, random forest, support vector machine, or probit regression model, etc.

According to some embodiments that employ a weighted approach, such as in the Examples as provided below, limma model is selected as the second statistical model. Further according to some embodiments, both first and second statistical models use limma model. As such, in the sub-step S210, when the differential expression analysis are carried out over the miRNA expression profiles, coefficients are generated for all miRNAs that are examined, and the coefficients corresponding to the at least one miRNA in the selected miRNA biomarker set based on which the diagnostic model is built can be used as the weights therefor in the diagnostic index calculation in the sub-step S220.

According to some other embodiments that employ a weighted approach, the second statistical model based on which the weights for each miRNAs in the selected miRNA biomarker set are obtained is different from the first statistical model based on which the miRNAs are ranked. For example, limma model is used as the first model, whereas LDA model is used as the second model.

According to some embodiments of the cancer diagnostic model development method as described above, the n miRNA(s) in the selected miRNA biomarker set are respectively the top n ranked miRNAs obtained in the differential expression analysis in the sub-step S210 as described above. In other words, the selected miRNA biomarker set comprises n selected miRNAs, which together substantially represent the top miRNAs with ranking of 1, 2, 3, . . . , n. In one illustrating example, the selected miRNA biomarker set may consist of 4 miRNAs, which respectively have rankings of 1, 2, 3, and 4 in the sub-step S210.

It is to be noted that the n miRNA(s) in the selected miRNA biomarker set do not necessarily respectively represent all of the top n ranked miRNAs, and may represent only a subset of these top n ranked miRNAs according to some other embodiments of the disclosure. In one illustrating example, the selected miRNA biomarker set may consist of 5 miRNAs, which respectively have rankings of 1, 2, 3, 4, and 6 in the sub-step S210.

According to some embodiments of the cancer diagnostic model development method, after the sub-step S220, the step S200 may optionally further comprise a sub-step of:

S230: Evaluating the performance of the diagnostic model through cross validation in the training set.

As used herein, the term “cross validation” refers to a way to treat the data in a given data set, and specifically as used in here, it means splitting the training set into N equal parts (thus called N-fold cross validation) with each part taking turns to be a validation set and the remaining parts together as a training set. A diagnostic model is developed in the training set using the same approach as described in S210 and S220, and then validated in the validation set. Herein, the fold number N of the cross validation can be any integer larger than or equal to 2 (i.e. N≥2, e.g. 2, 3, 4, 5, . . . , 10, 20, etc.).

There can be different parameter(s) based on which, the performance of the diagnostic model obtained in the sub-step S220 can be evaluated in the sub-step S230.

According to some embodiments, the substep S230 of evaluating the performance of the diagnostic model comprises: calculating area under curve (AUC) of receiver operating characteristic (ROC) curves, and evaluating the performance of the diagnostic model based thereon (i.e. based on the AUC). Herein in order to evaluate the performance of the diagnostic model based on the calculated AUC value, a preset cutoff (e.g. 0.80, 0.90, 0.95, 0.99 etc.) for the AUC can be used: if the AUC value thus calculated is greater than or equal to the preset cutoff, then the diagnostic model is determined to have a good performance; if, however, the AUC value thus calculated is lower than the preset cutoff, then the diagnostic model is determined to have a sub-ideal or bad performance.

Yet according to some other embodiments, the substep S230 of evaluating the performance of the diagnostic model comprises: calculating specificity and sensitivity of the diagnostic model, and evaluating the performance of the diagnostic model based thereon (based on the specificity and sensitivity). Herein in order to evaluate the performance of the diagnostic model based on the calculated sensitivity and specificity values, a preset cutoff for the sensitivity (e.g. 0.80, 0.90, 0.95, 0.99, etc.) for a given specificity (e.g. a preset value of 0.90, 0.95, 0.98, or 0.99, etc.) can be used: if the sensitivity value for a given specificity thus calculated is greater than or equal to the preset cutoff, then the diagnostic model is determined to have a good performance; if, however, the sensitivity value for a given specificity thus calculated is lower than the preset cutoff, then the diagnostic model is determined to have a sub-ideal or bad performance.

It is noted that in addition to the use of AUC of ROC curves and to the use of specificity and sensitivity, there are other parameters that can be used to evaluate the performance of the diagnostic model thus obtained in the sub-step S220 of the cancer diagnostic model development method. Examples of these other parameters include, but are not limited to, overall accuracy, positive likelihood ratio, negative likelihood ratio, positive predictive value, negative predictive value, Youden's J, etc.

It is further noted that the sub-step S230 of evaluating the performance of the diagnostic model through cross validation in the training set is only optional and may be skipped according to some embodiments of the disclosure.

By means of the step S300, the diagnostic model thus obtained in the step S200 (and in particular in the sub-step S220) as described above is further validated using a validation set that is distinct from the training set with no overlapping subjects or patients. Herein the validation set comprises expression profiles of the selected miRNA biomarker set that are obtained from non-cancer subjects (i.e. control) and cancer patients having the cancer of interest (i.e. case).

More specifically, the step S300 can be carried out by assessing the same or different parameter(s) as used in the aforementioned sub-step S230 of evaluating the performance of the diagnostic model through cross validation in the training set. According to some embodiments of the disclosure, the AUC of ROC curves is used for the validation, such that when the AUC is greater than or equal to a preset cutoff (e.g. 0.80, 0.90, 0.95, 0.99, etc.), the diagnostic model is validated, or otherwise when the AUC is lower than the preset cutoff. Herein the preset cutoff for the AUC used in the step S300 can be same as or different from the preset cutoff for the AUC used in the sub-step S230 as described above. According to some other embodiments of the disclosure, sensitivity and specificity are used for the validation, such that when the sensitivity value for a given specificity (e.g. 0.90, 0.95, 0.98, or 0.99, etc.) thus calculated is greater than or equal to a preset cutoff (e.g. 0.80, 0.90, 0.95, 0.99, etc.), then the diagnostic model is validated, or otherwise when the sensitivity value for the given specificity thus calculated is lower than the preset cutoff. There are other parameters that can be used to validate the diagnostic model as well.

This disclosure further provides a computerized solution, which substantially serves, in a computerized and automatic manner, to implement the various steps and/or sub-steps of the cancer diagnostic model development method as described above. Such a computerized solution may be applied in a situation where the implementation of the various steps S100-S300, and furthermore the various sub-steps S210-S230 of step S200, of the cancer diagnostic model development method described above is automated by running a software program comprising program instructions in a computer, which brings about advantages such as high efficiency and great convenience.

Specifically, such a computerized solution may include a computerized system or computer system (or short as a “system”). The system comprises a collection of hardware (e.g. processor, memory, I/O interface, storage medium, etc.) and software (i.e. computer programs, including operation system software, and specific program software, etc.), which are configured to collaboratively work so as to collectively implement all or some steps of the cancer detection diagnostic model development method as described above. According to some embodiments, the system comprises a processor (i.e. controller) and a computer-readable non-transitory storage medium that is communicatively coupled to the processor. The non-transitory storage medium is configured to contain a software (i.e. program instructions) for execution by the processor, and the program instructions are configured to cause the processor to execute the various different steps and sub-steps in the cancer diagnostic model development method as described above.

As used herein and throughout the disclosure, the “processor” is interpreted to be exchangeable with “central controller” or “central computing unit (CPU)”, and can be deemed to be a single core or multi core processor, or a plurality of processors for parallel processing. The term “non-transitory,” as used herein, is intended to describe a tangible computer-readable storage medium excluding propagating electromagnetic signals, but are not intended to otherwise limit the type of physical computer-readable storage device that is encompassed by the phrase. Examples may include any tangible or non-transitory storage media or memory media such as electronic, magnetic, or optical media (e.g., disk or CD/DVD-ROM), or non-volatile memory storage (e.g., “flash” memory), etc.

As illustrated in FIG. 2, the system 100 can, in addition to the processor 10 and the computer-readable non-transitory storage medium 20, further comprise a bus 30, a memory 40, an I/O interface 50, and a communication interface 60. The processor 10, the storage medium 20, the memory 40, the I/O interface 50 and the communication interface 60 are all communicatively coupled with one another through the bus 30.

The storage medium 20 stores computer-executable program instructions which, when executed by the processor 10, cause the processor 10 to execute steps (1)-(3) of the method as described above. The memory 40 is configured to transiently store the program instructions obtained from the storage medium 20, and the processor 10 is configured to execute the program instructions transiently stored in the memory 40. The I/O interface 50 allows an input/output between the system 100 and a user, realizing the control of the system 100. The communication interface 60 can allow the system 100 to be communicatively connected to another computing device to exchange data. It is to be noted that these computer hardware components can be locally arranged, or can be remotely arranged through a network, such as an intranet, an internet, or a cloud.

In a second aspect, this disclosure further provides a method for detecting a cancer of interest from a subject (e.g. human). Herein the cancer detection method is substantially by means of the diagnostic model that is developed by the cancer detection diagnostic model according to any embodiment as described above.

Specifically, the cancer detection method comprises the following major steps:
S1000: determining an expression profile of the selected miRNA biomarker set from a biological sample obtained from the subject;
S2000: calculating a diagnostic index of the biological sample based on the expression profile of the selected miRNA biomarker set, wherein the diagnostic index is calculated based on formula:

$$diagnostic index = {\sum }_{i = 1}^{n} {t}_{i} * {miRNA}_{i} ; \qquad ( I )$$

Herein n is the total number of the at least one miRNA in the selected miRNA biomarker set, miRNAᵢ is the expression level of i^{th} miRNA in the selected miRNA biomarker set, i is an integer greater than zero and smaller than or equal to n; and tᵢ is a weight for the i^{th} miRNA; and
S3000: classifying the subject as having the cancer of interest or not based on the calculated diagnostic index, wherein the subject is classified as having the cancer of interest if the calculated diagnostic index is greater than or equal to a pre-determined threshold or as not having the cancer of interest if otherwise.

Herein in the step S1000 of the cancer detection method provided herein, the “biological sample” from the subject can be a liquid biopsy sample such as a blood sample, a serum sample, a plasma sample, a urine sample, a saliva sample, or a sputum sample, but can optionally be a sample from a tumor tissue. The “selected miRNA biomarker set” is substantially the miRNA biomarker set that has been selected when developing the diagnostic model for detecting the cancer of interest by means of the cancer detection diagnostic model development method as provided in the first aspect of the disclosure. The determination of the expression profile of the selected miRNA biomarker set from the biological sample obtained from the subject can be realized by means of a variety of probe-based approaches including Northern Blotting, microarray analysis, RNA-sequencing, or RNA in-situ hybridization, or by means of a variety of amplification-dependent approaches including reverse-transcription PCR (RT-PCR), quantitative RT-PCR (qRT-PCR), or digital RT-PCR. As used herein, each of the above miRNA detection approaches is to be understood within the common definition well-appreciated by people of ordinary skills in the field.

In the step S2000 of the cancer detection method provided herein, the diagnostic index can be calculated in a manner that is same as in the cancer detection diagnostic model development method as described above in the first aspect of the disclosure.

In the step S3000, the term “pre-determined threshold” refers to as a cut-point value of the diagnostic index that can be used to judge or determine with a given specificity/sensitivity if a subject has the cancer of interest or not. It is typically pre-determined based on an existing dataset comprising a range of diagnostic index values that have been obtained and calculated for an existing population of subjects known to have, and/or known to be absent of, the disease. International Patent Application No. WO2022261039A2 provides greater details for how to classify the subject as having the cancer of interest or not based on the calculated diagnostic index, whose disclosure is incorporated herein by reference in its entirety.

According to some embodiments, each miRNA in the selected miRNA biomarker set is from the top 200 miRNAs as listed in TABLE 1 provided in Example 1 below, and the total number of miRNAs in the selected miRNA biomarker set (i.e. “n”) is smaller than or equal to 200. It is to be noted that each miRNA in the selected miRNA biomarker set can be from any number of top miRNAs as ranked in the sub-step S210 of the step S200 of the cancer detection diagnostic model development method as described above, which can, depending on different embodiments, be top 100, top 50, or top 500, etc.

According to some embodiments that are preferred, the diagnostic index in step S2000 of the cancer detection method is calculated using weights from the limma model. It is to be noted that according to some other embodiments, the diagnostic index can alternatively and optionally be calculated via weights from other statistical models such as logistic regression model, LDA model, conditional logistic regression model, lasso regression model, ridge regression model, random forest, support vector machine, or probit regression model. It is to be further noted that the diagnostic index can alternatively and optionally be calculated via an unweighted approach, whereby the weight for each miRNA in the selected miRNA biomarker set is an equal number (e.g. 1, 2, 0.5, etc.). In other word, for any miRNA; in the miRNA biomarker set, tᵢ=C (i.e. constant number).

According to some embodiments of the cancer detection method, the n miRNA(s) in the selected miRNA biomarker set are respectively the top n ranked miRNAs obtained in the differential expression analysis in the sub-step S210 of the step S200 of the cancer detection diagnostic model development method as described above in the first aspect of the disclosure. In other words, the selected miRNA biomarker set comprises n selected miRNAs, which together substantially represent the top miRNAs with ranking of 1, 2, 3, . . . , n. It is to be noted that the n miRNA(s) in the selected miRNA biomarker set do not necessarily respectively represent all of the top n ranked miRNAs, and may represent only a subset of these top n ranked miRNAs according to some other embodiments of the disclosure.

Herein according to some embodiments of the cancer detection method as provided herein, n is no less than 4 and no more than 200 (4≤n≤200), and the classification is capable of achieving an AUC of more than approximately 0.97 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer.

According to some embodiments, n is no less than 4 and no more than 200 (4≤n≤200), and the classification is capable of achieving a sensitivity of at least approximately 0.75 while maintaining a specificity of no less than approximately 0.99 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer.

Further according to some embodiments, the classification is capable of achieving a sensitivity of at least approximately 0.80 while maintaining a specificity of no less than approximately 0.99 for detecting lung cancer, colorectal cancer, esophageal cancer, gastric cancer, liver cancer, ovarian cancer, pancreatic cancer, biliary tract cancer, bladder cancer, glioma, or prostate cancer.

Further according to some embodiments, the classification is capable of achieving a sensitivity of at least approximately 0.90 while maintaining a specificity of no less than approximately 0.99 for detecting lung cancer, colorectal cancer, esophageal cancer, gastric cancer, pancreatic cancer, biliary tract cancer, bladder cancer, glioma, or prostate cancer.

Further according to some embodiments, the classification is capable of achieving a sensitivity of at least approximately 0.95 while maintaining a specificity of no less than approximately 0.99 for detecting lung cancer, esophageal cancer, gastric cancer, biliary tract cancer, bladder cancer, glioma, or prostate cancer.

Further according to some embodiments, the classification is capable of achieving a sensitivity of at least approximately 0.99 while maintaining a specificity of no less than approximately 0.99 for detecting lung cancer, esophageal cancer, gastric cancer, biliary tract cancer, bladder cancer, or prostate cancer.

According to some embodiments of the cancer detection method as provided herein, the selected miRNA biomarker set consists of top 4 miRNAs including hsa-miR-5100, hsa-miR-1228-5p, hsa-miR-8073, and hsa-miR-663a. As such, the classification is capable of achieving a sensitivity of at least approximately 0.75 while maintaining a specificity of no less than approximately 0.99 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer. Further optionally, the classification is capable of achieving a sensitivity of at least approximately 0.80 while maintaining a specificity value of approximately 0.99 for detecting lung cancer, colorectal cancer, esophageal cancer, gastric cancer, liver cancer, pancreatic cancer, biliary tract cancer, bladder cancer, glioma, or prostate cancer. Further optionally, the classification is capable of achieving a sensitivity of at least approximately 0.90 while maintaining a specificity value of approximately 0.99 for detecting lung cancer, colorectal cancer, esophageal cancer, gastric cancer, pancreatic cancer, biliary tract cancer, bladder cancer, glioma, or prostate cancer. Further optionally, the classification is capable of achieving a sensitivity of at least approximately 0.95 while maintaining a specificity value of approximately 0.99 for detecting lung cancer, gastric cancer, biliary tract cancer, bladder cancer, glioma, or prostate cancer. Further optionally, the classification is capable of achieving a sensitivity of at least approximately 0.99 while maintaining a specificity value of approximately 0.99 for detecting lung cancer, gastric cancer, biliary tract cancer, or bladder cancer.

According to some embodiments of the cancer detection method as provided herein, the selected miRNA biomarker set consists of top 5 miRNAs including hsa-miR-5100, hsa-miR-1228-5p, hsa-miR-8073, hsa-miR-663a, hsa-miR-320a, and the classification is capable of achieving a sensitivity of at least approximately 0.80 while maintaining a specificity value of approximately 0.99 for detecting lung cancer, colorectal cancer, esophageal cancer, gastric cancer, liver cancer, ovarian cancer, pancreatic cancer, biliary tract cancer, bladder cancer, glioma, or prostate cancer.

According to some embodiments of the cancer detection method as provided herein, the selected miRNA biomarker set consists of top 10 miRNAs from TABLE 1, and the classification is capable of achieving a sensitivity of at least approximately 0.99 while maintaining a specificity value of approximately 0.99 for detecting gastric cancer, esophageal cancer, biliary tract cancer, or prostate cancer.

According to some embodiments of the cancer detection method as provided herein, the selected miRNA biomarker set consists of top 15 miRNAs from TABLE 1, and the classification is capable of achieving a sensitivity of at least approximately 0.90 while maintaining a specificity value of approximately 0.99 for detecting lung cancer, esophageal cancer, gastric cancer, liver cancer, pancreatic cancer, biliary tract cancer, bladder cancer, glioma, or prostate cancer.

In any embodiment of the cancer detection method as described above, the method optionally further comprises a step of performing an evaluation of the subject, wherein said evaluation comprises a diagnosis of the cancer or a detection of a recurrence of the cancer. Herein, the phrase “diagnosis of the cancer” refers to as the detection of the cancer in a subject previously known not to have the cancer, whereas the phrase “recurrence of the cancer” refers to as the detection of the cancer again in a subject with the cancer who has previously been treated to remove the cancer to become cancer-free.

In any embodiment of the method as described above, the method optionally further comprises a step of performing a diagnostic procedure on the subject when the subject is classified as having the cancer. Herein the diagnostic procedure may optionally comprise physical examination, pathology examination of a biopsy from the subject, immunohistochemistry examination, or imaging examination such as x-rays, computed tomography (CT), ultrasonography, and/or magnetic resonance imaging.

In any embodiment of the method as described above, the method optionally further comprises a step of administering to the subject a therapeutic regimen when the subject is classified as having the cancer. Herein, a variety of known therapeutic regimens can be administered in the method, which include surgery, radiotherapy, chemotherapy, hormonal therapy, targeted therapy, immunotherapy or the combination thereof. These above therapeutic regimens have been well-established for each different cancer mentioned above.

This disclosure further provides a computerized solution, including a computerized system (including a processor and a non-transitory storage medium) for implementing the various steps and/or sub-steps of the cancer detection method as described above. Details for such computerized solution can reference to the computerized solution for the cancer detection diagnostic model development method as provided in the first aspect of the disclosure.

This disclosure further provides a kit for detecting a cancer from a biological sample obtained from a subject, which is substantially employed for implementing the cancer detection method as described above.

As used herein, and elsewhere in the disclosure as well, the term “kit” refers to as a collection of articles and/or instructions. An article included in the kit can be a physical entity or a component thereof. Examples of articles that can be included in the kit as disclosed herein can include one or more nucleic acids (e.g. polynucleotides), or one or more device, apparatus or equipment (e.g. a molecular array or microarray that comprises the one or more nucleic acids). An instruction included in the kit can be a description of the specific steps to be performed (e.g. a manual), which can be printed on a physical medium (e.g. paper, card, etc.), on a computer-readable storage medium (e.g. hard disc, compact disc or CD, flash drive, etc.), or even stored in the internet (e.g. in an accessible cloud space), etc.

The kit can comprise at least the following components (1) and (2) (i.e. articles and/or instructions):

Component (1): at least one nucleic acid, each capable of specifically recognizing each miRNA in the selected miRNA biomarker set to thereby allow an expression profile of the selected miRNA biomarker set to be obtained from the biological sample. Herein the selected miRNA biomarker set is substantially the miRNA biomarker set that has been selected when developing the diagnostic model for detecting the cancer of interest by means of the cancer detection diagnostic model development method as provided in the first aspect of the disclosure.

Component (2): at least one instruction, comprising a first instruction and a second instruction. Herein the first instruction is configured to substantially implement the step S2000 of calculating the diagnostic index of the biological sample based on the expression profile of the selected miRNA biomarker set, and the second instruction is configured to substantially implement the step S3000 of classifying the subject as having the cancer or not, the subject is classified as having the cancer if the calculated diagnostic index is greater than or equal to a pre-determined threshold or as not having the cancer if otherwise.

Herein, in component (1) of the kit, the at least one nucleic acid can optionally comprise a polynucleotide capable of specifically hybridizing under a stringent condition to: either (a) a polynucleotide comprising or consisting of a nucleotide sequence of each miRNA in the selected miRNA biomarker set, a derivative thereof, a variant thereof having at least 80% sequence identity, or a fragment thereof comprising 15 or more consecutive nucleotides; or (b) a polynucleotide comprising or consisting of a nucleotide sequence complementary to a nucleotide sequence of each miRNA in the selected miRNA biomarker set, a derivative thereof, a variant thereof having at least 80% sequence identity, or a fragment thereof comprising 15 or more consecutive nucleotides.

According to different embodiments, the at least one instruction in component (2) in the kit may further comprise a third instruction for performing an evaluation of the subject, wherein said evaluation comprises a diagnosis of the cancer or a detection of a recurrence of the cancer; or may further comprise a fourth instruction for administering to the subject a therapeutic regimen when the subject is classified as having the cancer.

According to some embodiments, the at least one instruction in component (2) in the kit may further comprise a first additional instruction for obtaining the expression profile of the selected miRNA biomarker set, comprising a procedure for performing Northern Blotting, microarray analysis, RNA-sequencing, or RNA in-situ hybridization by means of the at least one nucleic acid. Herein, the at least one nucleic acid may optionally be arranged on a molecular array.

According to some embodiments, the kit may further comprise at least one set of amplification primers, each set capable of specifically amplifying each of the at least one miRNA in the selected miRNA biomarker set from the biological sample. As such, the at least one instruction in component (2) in the kit may further comprise a second additional instruction for obtaining the expression profile of the selected miRNA biomarker set, comprising a procedure for performing reverse-transcription PCR (RT-PCR), quantitative RT-PCR (qRT-PCR), or digital RT-PCR by means of the at least one nucleic acid and the at least one set of amplification primers.

In the following, one example (i.e., Example 1) is provided to illustrate the inventions as described above in the various aspects of the disclosure.

### Example 1

In this example, by using eight publicly available serum miRNA microarray datasets totaling 6,283 cancer patients and 5,130 non-cancer controls, a large multi-cancer training set (“Train Set” hereinafter) that includes multiple cancer types, rather than a single cancer type, was constructed. Differentially expressed miRNAs were identified and 10-fold cross validation was used to determine the optimal number of miRNAs for building a diagnostic model in the training set. The performance of the model was then evaluated in the three validation sets. More detailed information is provided below.

### Methods

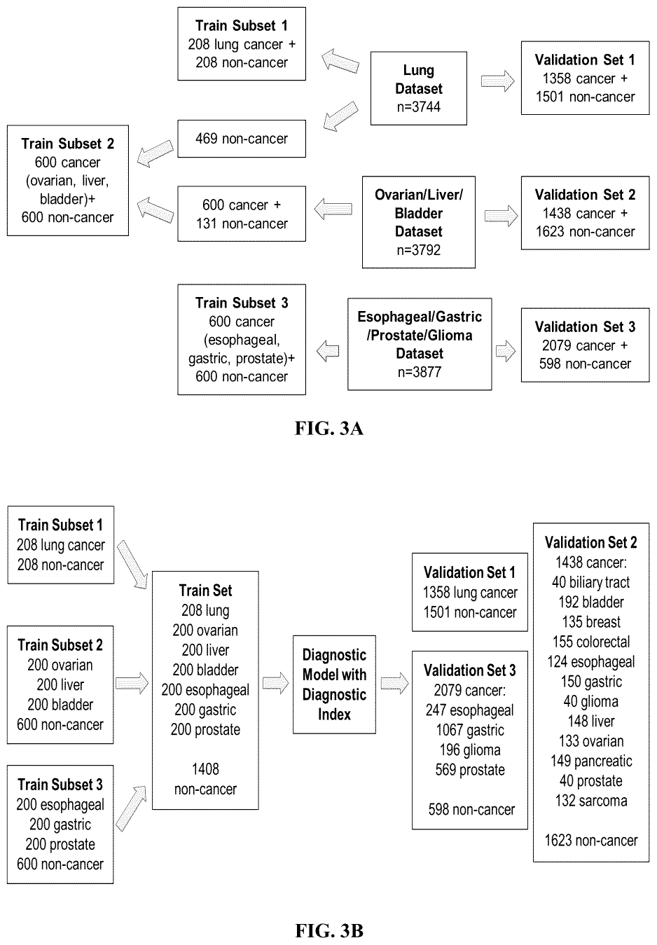

Study design and construction of train and validation datasets: Eight serum miRNA microarray datasets from Gene Expression Omnibus (GEO) (Zhang A et al., 2022; Zhang A and Hu H, 2022) were identified. They were all generated from the Japanese nationwide research project “Development and Diagnostic Technology for Detection of miRNA in Body Fluids” that was designed to characterize serum miRNAs in over 50,000 participants across 13 cancer types using a standardized microarray platform. These eight datasets were originally used to develop individual diagnostic models for lung (GSE137140) (Asakura K, et al., 2020), ovarian (GSE106817) (Yokoi A, et al., 2018), liver (GSE113740) (Yamamoto Y, et al., 2020), bladder (GSE113486) (Usuba W, et al., 2019), esophageal squamous cell (GSE122497) (Sudo K, et al., 2019), gastric (GSE164174) (Abe S, et al., 2021), prostate (GSE112264) (Urabe F, et al., 2019) and glioma (GSE139031) (Ohno M, et al., 2019) cancers, respectively. After removing redundant cases, three large datasets were assembled that were independent of each other: a lung cancer dataset (n=3744) (Zhang A et al., 2022; Asakura K, et al., 2020), a combined dataset by merging the ovarian, liver and bladders cancer datasets (n=3792) (Zhang A et al., 2022; Yokoi A, et al., 2018; Yamamoto Y, et al., 2020; Usuba W, et al., 2019), and a combined dataset by merging the esophageal squamous cell, gastric, prostate and glioma cancer datasets (n=3877) (Zhang A and Hu H, 2022; Sudo K, et al., 2019; Abe S, et al., 2021; Ohno M, et al., 2019; Urabe F, et al., 2019). Based on these three large datasets, a large training set was constructed that included 1408 cancer patients from 7 cancer types (208 lung cancer patients and 200 patients each for ovarian, liver, bladder, esophageal, gastric, and prostate) and 1408 age- and gender-matched non-cancer controls for the development of a diagnostic model for detecting multiple cancer types. All the remaining cases formed three separate independent validation sets (FIGS. 3A and 3B).

Blood sample collection and miRNA microarray analysis: Collection of blood serum samples and microarray expression analysis had been previously described (Asakura K et al., 2020). Briefly, total RNA was extracted from 300 μl serum collected from cancer patients prior to surgery and from non-cancer controls, labeled by 3DGene® miRNA Labeling kit and hybridized to 3D-Gene® Human miRNA Oligo Chip (Toray Industries, Kanagawa, Japan). The signal intensities for miRNAs were determined after background subtraction and normalization according to three pre-selected internal control miRNAs (miR-149-3p, miR-2861, and miR-4463).

Diagnostic model development: miRNA biomarker identification and all model development work were done in the multi-cancer Train Set only. The differential miRNA expression between cancer vs non-cancer was evaluated using Linear Model for Microarray Data (limma) (Ritchie M E, et al., 2015). miRNAs were then ranked based on the statistical significance of differential expression and the top miRNAs were used to build diagnostic models for distinguishing cancer vs. non-cancer. A diagnostic index was calculated for each diagnostic model as a linear sum of expression levels of the selected miRNAs weighted by limma statistics. Ten-fold cross validation was performed to determine the optimal number of miRNAs to be included in the final diagnostic model that had the highest area-under-the-curve (AUC) of the Receiver Operating Characteristics (ROC) curves for distinguishing cancer vs. non-cancer. The cut-point for the diagnostic index was chosen to ensure at least 99% specificity (i.e., <1% false positive rate) as the model may potentially be used as a screening tool in at-risk general public.

Diagnostic model validation: The three independent validation datasets contained mutually exclusive samples that were not used in model development, with each offering distinct characteristics for the validation of the developed model. Validation Set 1 not only was of a very large sample size for lung cancer cases, but also contained comprehensive patient-level clinicopathologic data in contrast to the other two validation datasets, making it possible to assess model performance on early-stage cancers and different histology subtypes. Validation Set 2 contained samples from 12 other cancer types, thus expanding the evaluation of model performance across multiple cancer types. Validation Set 3 comprised large numbers of the cases from four cancer types including the two cancer types with low sample size in Validation Set 2, allowing additional independent verification of the model performance.

Statistical analysis: AUC of the ROC curve analysis, sensitivity, and specificity were used to measure the diagnostic performance for detecting cancer vs. non-cancer. Sensitivity was defined as the proportion of cancer patients who were correctly identified as cancer by the diagnostic model, while specificity was defined as the proportion of non-cancer participants who were correctly identified as non-cancer. limma analysis was performed using Bioconductor package limma (Ritchie M E, et al., 2015). All statistical analysis was conducted using R version 4.2.1.

### Results

Participants and Datasets: Detailed demographic and clinical information for those cancer types of large sample size were described in the original publications. Briefly, the patients in the lung cancer dataset (n=1566) had mean age 65 y, composed of 57% male and 62% former or current smokers, with 78% of the tumors being adenocarcinoma, 14% squamous carcinoma, 87% stage I or II. The bladder cancer dataset (n=392) included patients of mean age 68 y, 72% male, 95% non-metastatic, 88% nodal-negative, 77% T1 and 80% high grade. The ovarian cancer dataset (n=333) included patients with mean age 57 y, 35% stage I or II, 96% epithelial (including 55%, 19% and 13% for serous, clear cell, and endometrioid histology, respectively). The patients in the liver cancer dataset (n=348) were of mean age 68 y, 78% male, and 70% stage I or II. The esophageal cancer dataset (n=447) consisted of patients with a mean age 67 y, 97% male, and 66% stage I or II. The gastric cancer dataset (n=1267) included patients with mean age 66 y, 77% male and all stage I or II. The glioma dataset (n=196) comprised patients with mean age 56 y and 57% were male. Finally, the patients in the prostate cancer dataset (n=769) had mean age 68 y, 93% node-negative, and 92% non-metastatic.

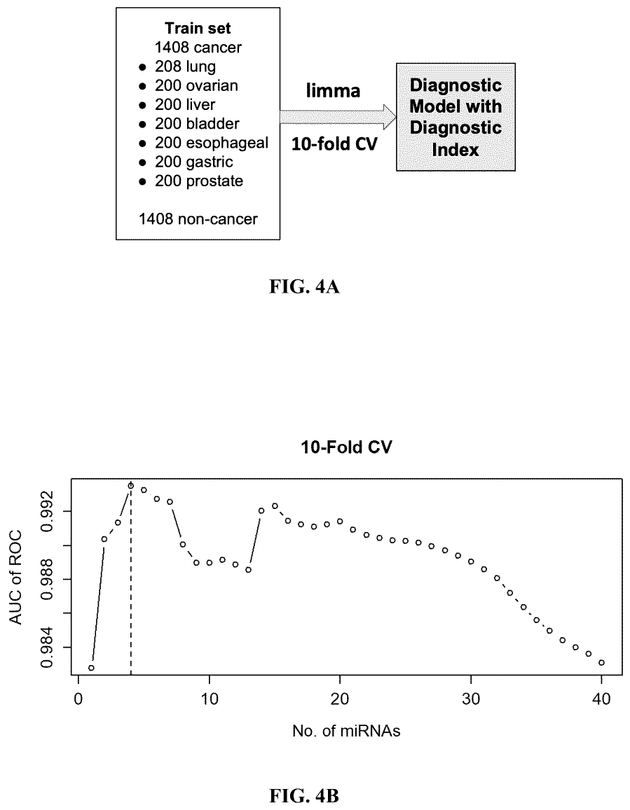

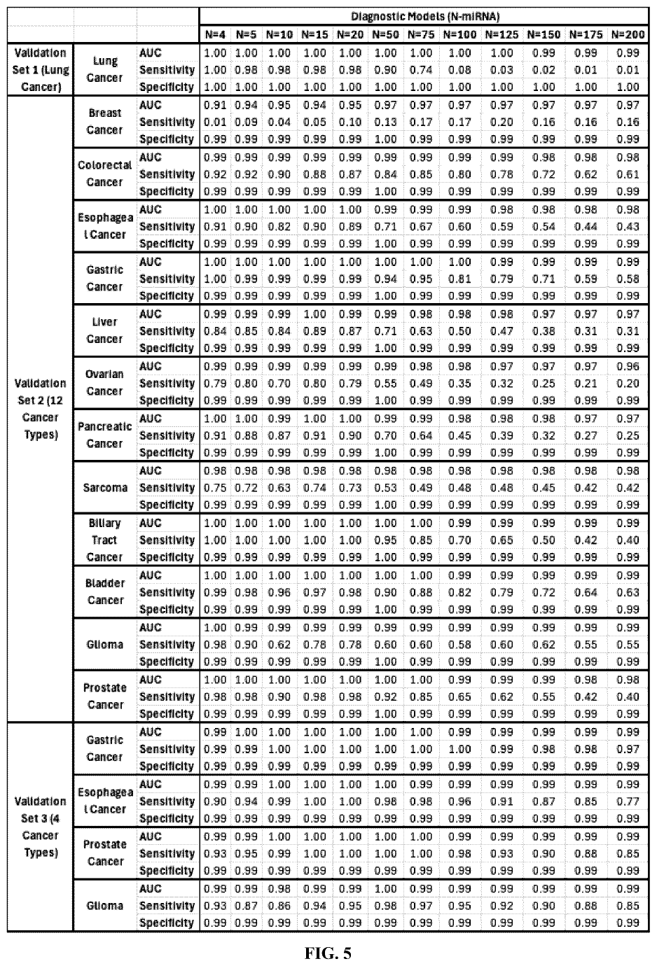

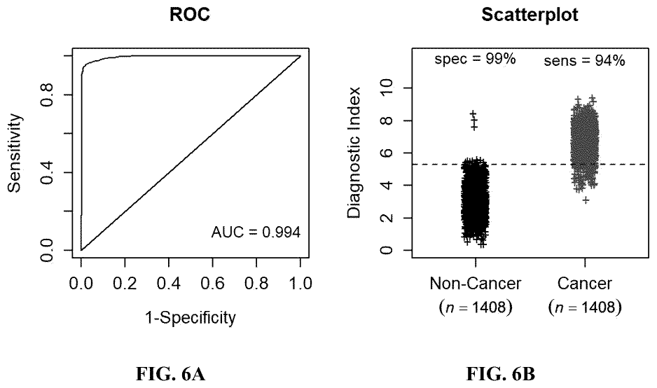

Cancer Diagnostic Model Development: All diagnostic model development work was performed in the multi-cancer Train Set, which included 1408 cancer patients and 1408 non-cancer controls matched by age and gender (FIG. 3B). First, limma analysis was used to assess the differential expression of miRNAs between cancer and non-cancer. miRNAs were then ranked based on the adjusted p values. The top 200 differentially expressed miRNAs are listed in Table 1. Then ten-fold cross validation using the multi-cancer Train Set was performed (FIG. 4A), and it was revealed that the top 4 miRNAs (hsa-miR-5100, hsa-miR-1228-5p, hsa-miR-8073 and hsa-miR-663a) provided the highest AUC in the ROC analysis and thus were included in the final diagnostic model (FIG. 4B). Then the diagnostic performances of different diagnostic models in their respective performances to detect different cancers using difference validation sets were compared. These different diagnostic models vary by the number N of top miRNAs (N is between 4 and 200), and the results are shown in FIG. 5. We calculated a diagnostic index by the weighted sum of the 4 miRNA expression levels and normalized to the range of 0 to 10. This 4-miRNA model achieved an AUC value of 0.994 within the Train Set (FIG. 6A). A cut-point of 5.3 was chosen to yield an overall >99% specificity (i.e., <1% false positives) across the non-cancer cases, and an overall 94% sensitivity (FIG. 6B). The AUC and sensitivity of the model for each of the 7 cancer types in the multi-cancer Train Set ranged from 0.985 and 84% for ovarian cancer to 0.998 and 100% for bladder and gastric cancers, respectively (FIG. 5).

TABLE 1
Top 200 miRNAs that have been identified through differential
expression analysis of the multi-cancer Train set.

| miRNA | miRBase | log Fold | adjusted | AUC of | | SEQ ID |
|---|---|---|---|---|---|---|
| name | Accession ID | Change | P value | ROC | Sequence | NO: |
| hsa-miR- | MIMAT0022259 | 3.5245 | 0.00E+00 | 0.9828 | UUCAGAUCCCAG | 1 |
| 5100 | | | | | CGGUGCCUCU | |
| hsa-miR- | MIMAT0005582 | −0.93 | 0.00E+00 | 0.0514 | GUGGGCGGGGGC | 2 |
| 1228-5p | | | | | AGGUGUGUG | |
| hsa-miR- | MIMAT0031000 | 2.3661 | 0.00E+00 | 0.9503 | ACCUGGCAGCAG | 3 |
| 8073 | | | | | GGAGCGUCGU | |
| hsa-miR- | MIMAT0003326 | 1.6038 | 0.00E+00 | 0.9479 | AGGCGGGGCGCC | 4 |
| 663a | | | | | GCGGGACCGC | |
| hsa-miR- | MIMAT0000510 | 2.2859 | 0.00E+00 | 0.9519 | AAAAGCUGGGUU | 5 |
| 320a | | | | | GAGAGGGCGA | |
| hsa-miR- | MIMAT0027468 | −1.223 | 0.00E+00 | 0.0715 | GCCGGGGCUUUG | 6 |
| 6784-5p | | | | | GGUGAGGG | |
| hsa-miR- | MIMAT0019957 | 2.0669 | 0.00E+00 | 0.9465 | GAUGCGCCGCCC | 7 |
| 4787-3p | | | | | ACUGCCCCGCGC | |
| hsa-miR- | MIMAT0019776 | 2.2883 | 0.00E+00 | 0.9418 | CUCCUGGGGCCC | 8 |
| 1343-3p | | | | | GCACUCUCGC | |
| hsa-miR- | MIMAT0027504 | 1.0855 | 0.00E+00 | 0.9262 | CUAGGUGGGGGG | 9 |
| 6802-5p | | | | | CUUGAAGC | |
| hsa-miR- | MIMAT0005792 | 2.9231 | 0.00E+00 | 0.956 | AAAAGCUGGGUU | 10 |
| 320b | | | | | GAGAGGGCAA | |
| hsa-miR- | MIMAT0019229 | −0.935 | 0.00E+00 | 0.0787 | GUGGGUUGGGGC | 11 |
| 3940-5p | | | | | GGGCUCUG | |
| hsa-miR- | MIMAT0007347 | 1.2063 | 0.00E+00 | 0.9354 | CUCGGCGCGGGG | 12 |
| 1469 | | | | | CGCGGGCUCC | |
| hsa-miR- | MIMAT0016879 | 1.8502 | 0.00E+00 | 0.932 | CCCCGCCACCGC | 13 |
| 4258 | | | | | CUUGG | |
| hsa-miR- | MIMAT0005880 | 5.3117 | 0.00E+00 | 0.9799 | UGGAUUUUUGGA | 14 |
| 1290 | | | | | UCAGGGA | |
| hsa-miR- | MIMAT0019947 | 2.4921 | 0.00E+00 | 0.903 | CCCCGGUGUUGG | 15 |
| 4783-3p | | | | | GGCGCGUCUGC | |
| hsa-miR- | MIMAT0015041 | 1.6889 | 0.00E+00 | 0.922 | AUCCCACCACUG | 16 |
| 1260b | | | | | CCACCAU | |
| hsa-miR- | MIMAT0027430 | −0.651 | 0.00E+00 | 0.0823 | GUGAGGCGGGGC | 17 |
| 6765-5p | | | | | CAGGAGGGUGUG | |
| | | | | | U | |
| hsa-miR- | MIMAT0022947 | 1.7056 | 0.00E+00 | 0.9201 | GUGAGUGGGAGC | 18 |
| 1238-5p | | | | | CCCAGUGUGUG | |
| hsa-miR- | MIMAT0000071 | 4.1887 | 0.00E+00 | 0.9507 | ACUGCAGUGAAG | 19 |
| 17-3p | | | | | GCACUUGUAG | |
| hsa-miR- | MIMAT0019757 | 1.3999 | 0.00E+00 | 0.8943 | GGGGCUGUGAUU | 20 |
| 4675 | | | | | GACCAGCAGG | |
| hsa-miR- | MIMAT0018976 | 2.221 | 0.00E+00 | 0.9205 | GGAUCCGAGUCA | 21 |
| 4454 | | | | | CGGCACCA | |
| hsa-miR- | MIMAT0018444 | 1.503 | 0.00E+00 | 0.9083 | AGACACAUUUGG | 22 |
| 642b-3p | | | | | AGAGGGACCC | |
| hsa-miR- | MIMAT0019071 | 2.1278 | 0.00E+00 | 0.8941 | CCCCGGGGAGCC | 23 |
| 4532 | | | | | CGGCG | |
| hsa-miR- | MIMAT0019806 | 1.1253 | 0.00E+00 | 0.9101 | AGCGGGGAGGAA | 24 |
| 4706 | | | | | GUGGGCGCUGCU | |
| | | | | | U | |
| hsa-miR- | MIMAT0027462 | −0.676 | 0.00E+00 | 0.0828 | CGGGCCGGAGGU | 25 |
| 6781-5p | | | | | CAAGGGCGU | |
| hsa-miR- | MIMAT0027500 | −0.954 | 0.00E+00 | 0.0695 | GUAGGUGACAGU | 26 |
| 6800-5p | | | | | CAGGGGCGG | |
| hsa-miR- | MIMAT0004679 | 1.7872 | 0.00E+00 | 0.9031 | GAGGGUUGGGUG | 27 |
| 296-3p | | | | | GAGGCUCUCC | |
| hsa-miR- | MIMAT0005922 | −0.618 | 0.00E+00 | 0.0979 | CGGGCGUGGUGG | 28 |
| 1268a | | | | | UGGGGG | |
| hsa-miR- | MIMAT0019852 | −1.982 | 0.00E+00 | 0.1077 | CUGGCGGAGCCC | 29 |
| 4730 | | | | | AUUCCAUGCCA | |
| hsa-miR- | MIMAT0005951 | 2.3676 | 0.00E+00 | 0.8873 | ACUCGGCGUGGC | 30 |
| 1307-3p | | | | | GUCGGUCGUG | |
| hsa-miR- | MIMAT0022943 | 1.5602 | 0.00E+00 | 0.9012 | AGUGGGAGGCCA | 31 |
| 1233-5p | | | | | GGGCACGGCA | |
| hsa-miR- | MIMAT0022742 | 1.6229 | 0.00E+00 | 0.9101 | ACCACUGCACUC | 32 |
| 1273g-3p | | | | | CAGCCUGAG | |
| hsa-miR- | MIMAT0015072 | 4.4929 | 0.00E+00 | 0.9052 | AAAGCUGGGUUG | 33 |
| 320e | | | | | AGAAGG | |
| hsa-miR- | MIMAT0003282 | 2.3584 | 0.00E+00 | 0.8832 | GAACGCCUGUUC | 34 |
| 614 | | | | | UUGCCAGGUGG | |
| hsa-miR- | MIMAT0004697 | 4.7005 | 0.00E+00 | 0.887 | UCGAGGAGCUCA | 35 |
| 151a-5p | | | | | CAGUCUAGU | |
| hsa-miR- | MIMAT0016916 | 1.7899 | 0.00E+00 | 0.8966 | ACCCCACUCCUG | 36 |
| 4286 | | | | | GUACC | |
| hsa-miR- | MIMAT0000725 | 1.2212 | 0.00E+00 | 0.9018 | ACUCAAAAUGGG | 37 |
| 373-5p | | | | | GGCGCUUUCC | |
| hsa-miR- | MIMAT0027623 | 1.003 | 0.00E+00 | 0.8868 | ACUGGGUAGGUG | 38 |
| 6861-5p | | | | | GGGCUCCAGG | |
| hsa-miR- | MIMAT0018929 | 0.489 | 0.00E+00 | 0.9076 | GGUGGGCUUCCC | 39 |
| 4417 | | | | | GGAGGG | |
| hsa-miR- | MIMAT0021021 | 0.9201 | 0.00E+00 | 0.8909 | AGGGCUGGACUC | 40 |
| 5001-5p | | | | | AGCGGCGGAGCU | |
| hsa-miR- | MIMAT0019774 | 1.2096 | 0.00E+00 | 0.9162 | CAGCCCUCCUCC | 41 |
| 4687-5p | | | | | CGCACCCAAA | |
| hsa-miR- | MIMAT0000440 | 4.9936 | 0.00E+00 | 0.899 | CAACGGAAUCCC | 42 |
| 191-5p | | | | | AAAAGCAGCUG | |
| hsa-miR- | MIMAT0005898 | 5.8537 | 0.00E+00 | 0.8895 | AAUGGAUUUUUG | 43 |
| 1246 | | | | | GAGCAGG | |
| hsa-miR- | MIMAT0019791 | 0.7797 | 0.00E+00 | 0.9245 | AGGGGGCGCAGU | 44 |
| 4697-5p | | | | | CACUGACGUG | |
| hsa-miR- | MIMAT0022694 | 1.3342 | 0.00E+00 | 0.8842 | GCAGGGACAGCA | 45 |
| 211-3p | | | | | AAGGGGUGC | |
| hsa-miR- | MIMAT0031180 | 1.6903 | 0.00E+00 | 0.9003 | UUCCCAGCCAAC | 46 |
| 7977 | | | | | GCACCA | |
| hsa-miR- | MIMAT0019223 | 4.7018 | 0.00E+00 | 0.8764 | AGUGGCAAAGUC | 47 |
| 3688-5p | | | | | UUUCCAUAU | |
| hsa-miR- | MIMAT0019052 | 2.8801 | 0.00E+00 | 0.8934 | AGGACUGGACUC | 48 |
| 4515 | | | | | CCGGCAGCCC | |
| hsa-miR- | MIMAT0021127 | 1.1135 | 0.00E+00 | 0.8899 | AUCCAGUUCUCU | 49 |
| 5195-3p | | | | | GAGGGGGCU | |
| hsa-miR- | MIMAT0000100 | 4.435 | 0.00E+00 | 0.89 | UAGCACCAUUUG | 50 |
| 29b-3p | | | | | AAAUCAGUGUU | |
| hsa-miR- | MIMAT0015064 | −2.092 | 0.00E+00 | 0.0916 | UGAGGGGCCUCA | 51 |
| 3184-5p | | | | | GACCGAGCUUUU | |
| hsa-miR- | MIMAT0005867 | 1.2394 | 0.00E+00 | 0.8879 | GGUGGCCCGGCC | 52 |
| 663b | | | | | GUGCCUGAGG | |
| hsa-miR- | MIMAT0019064 | 2.4656 | 0.00E+00 | 0.8823 | GGGGGGAUGUGC | 53 |
| 4525 | | | | | AUGCUGGUU | |
| hsa-miR- | MIMAT0019741 | 4.6402 | 0.00E+00 | 0.871 | AUACAUGUCAGA | 54 |
| 4666a-5p | | | | | UUGUAUGCC | |
| hsa-miR- | MIMAT0004687 | 0.911 | 0.00E+00 | 0.895 | ACUCAAACUGUG | 55 |
| 371a-5p | | | | | GGGGCACU | |
| hsa-miR- | MIMAT0027654 | 1.004 | 0.00E+00 | 0.8876 | AGGGCCGAAGGG | 56 |
| 6877-5p | | | | | UGGAAGCUGC | |
| hsa-miR- | MIMAT0001631 | 4.6194 | 2.05E-320 | 0.8959 | AAACCGUUACCA | 57 |
| 451a | | | | | UUACUGAGUU | |
| hsa-miR- | MIMAT0006764 | 3.8917 | 1.31E-319 | 0.8968 | AAAAGCUGGGUU | 58 |
| 320d | | | | | GAGAGGA | |
| hsa-miR- | MIMAT0027510 | −0.687 | 1.34E-315 | 0.125 | UAGGGGGCGGCU | 59 |
| 6805-5p | | | | | UGUGGAGUGU | |
| hsa-miR- | MIMAT0019069 | −0.856 | 1.51E-314 | 0.1413 | CCCAGCAGGACG | 60 |
| 4530 | | | | | GGAGCG | |
| hsa-miR- | MIMAT0018925 | −0.522 | 4.25E-314 | 0.1232 | CGGGCGUGGUGG | 61 |
| 1268b | | | | | UGGGGGUG | |
| hsa-miR- | MIMAT0000690 | 0.7536 | 1.34E-313 | 0.8845 | AGGGCCCCCCCU | 62 |
| 296-5p | | | | | CAAUCCUGU | |
| hsa-miR- | MIMAT0014977 | 2.7865 | 1.39E-310 | 0.8818 | AUAUGGGUUUAC | 63 |
| 3115 | | | | | UAGUUGGU | |
| hsa-miR- | MIMAT0018111 | 3.2347 | 0.00E+00 | 0.8826 | UGCGACAUUGGA | 64 |
| 3683 | | | | | AGUAGUAUCA | |
| hsa-miR- | MIMAT0027474 | 1.1852 | 3.72E-308 | 0.8836 | UGGCGGGGGUAG | 65 |
| 6787-5p | | | | | AGCUGGCUGC | |
| hsa-miR- | MIMAT0022706 | 3.2109 | 1.07E-307 | 0.8825 | AUCAAGGAUCUU | 66 |
| 561-5p | | | | | AAACUUUGCC | |
| hsa-miR- | MIMAT0019790 | 3.2421 | 1.03E-303 | 0.8796 | UGCAAGACGGAU | 67 |
| 4696 | | | | | ACUGUCAUCU | |
| hsa-miR- | MIMAT0027496 | −0.699 | 1.76E-303 | 0.1317 | CCAGGGGGAUGG | 68 |
| 6798-5p | | | | | GCGAGCUUGGG | |
| hsa-miR- | MIMAT0004945 | 1.0916 | 2.49E-303 | 0.8688 | UGCGGGGCUAGG | 69 |
| 744-5p | | | | | GCUAACAGCA | |
| hsa-miR- | MIMAT0018972 | 2.7276 | 7.06E-302 | 0.8791 | AAAAGUGAUUGC | 70 |
| 548ah-5p | | | | | AGUGUUUG | |
| hsa-miR- | MIMAT0027478 | 0.6762 | 8.03E-302 | 0.8792 | GUAGGGGCGUCC | 71 |
| 6789-5p | | | | | CGGGCGCGCGGG | |
| hsa-miR- | MIMAT0024615 | 3.7779 | 1.11E-301 | 0.8592 | GGCUGGUCAGAU | 72 |
| 6131 | | | | | GGGAGUG | |
| hsa-miR- | MIMAT0019869 | 2.8866 | 7.34E-301 | 0.8879 | AGGACUGAUCCU | 73 |
| 4740-5p | | | | | CUCGGGCAGG | |
| hsa-miR- | MIMAT0005793 | 2.8245 | 9.91E-301 | 0.904 | AAAAGCUGGGUU | 74 |
| 320c | | | | | GAGAGGGU | |
| hsa-miR- | MIMAT0003240 | −2.082 | 2.31E-300 | 0.0865 | GAGCCAGUUGGA | 75 |
| 575 | | | | | CAGGAGC | |
| hsa-miR- | MIMAT0019831 | 3.5082 | 4.54E-300 | 0.8848 | AGCUGUACCUGA | 76 |
| 4718 | | | | | AACCAAGCA | |
| hsa-miR- | MIMAT0018198 | 2.9612 | 1.75E-299 | 0.8803 | AACUAGUAAUGU | 77 |
| 3923 | | | | | UGGAUUAGGG | |
| hsa-miR- | MIMAT0019815 | 2.6091 | 1.57E-297 | 0.8738 | GGGUGAGGGCAG | 78 |
| 4710 | | | | | GUGGUU | |
| hsa-miR- | MIMAT0005914 | 2.9412 | 3.47E-296 | 0.878 | AUGGGUGAAUUU | 79 |
| 1262 | | | | | GUAGAAGGAU | |
| hsa-miR- | MIMAT0030986 | 1.769 | 8.87E-296 | 0.8583 | GGGGAACUGUAG | 80 |
| 8059 | | | | | AUGAAAAGGC | |
| hsa-miR- | MIMAT0019762 | 3.0913 | 2.84E-295 | 0.8765 | AAGGUAUUGUUC | 81 |
| 4678 | | | | | AGACUUAUGA | |
| hsa-miR- | MIMAT0027472 | −0.467 | 1.17E-293 | 0.1222 | GCGGUGGGGCCG | 82 |
| 6786-5p | | | | | GAGGGGCGU | |
| hsa-miR- | MIMAT0031095 | 1.1267 | 2.62E-293 | 0.8677 | GGGGGCCGAUAC | 83 |
| 128-2-5p | | | | | ACUGUACGAGA | |
| hsa-miR- | MIMAT0019805 | 3.5104 | 3.45E-293 | 0.8726 | UCAAUCACUUGG | 84 |
| 4705 | | | | | UAAUUGCUGU | |
| hsa-miR- | MIMAT0004922 | 2.8555 | 2.20E-292 | 0.8714 | UAUACCUCAGUU | 85 |
| 875-5p | | | | | UUAUCAGGUG | |
| hsa-miR- | MIMAT0022301 | 3.2719 | 7.50E-292 | 0.8734 | UGGGAGCUAAGC | 86 |
| 5591-5p | | | | | UAUGGGUAU | |
| hsa-miR- | MIMAT0015056 | 3.3173 | 2.29E-290 | 0.8701 | AGAAGGGGUGAA | 87 |
| 3179 | | | | | AUUUAAACGU | |
| hsa-miR- | MIMAT0019785 | 3.5817 | 1.53E-289 | 0.8759 | UGAGAGUGGAAU | 88 |
| 4693-3p | | | | | UCACAGUAUUU | |
| hsa-miR- | MIMAT0002883 | 3.7929 | 9.68E-289 | 0.8722 | AUUGACACUUCU | 89 |
| 514a-3p | | | | | GUGAGUAGA | |
| hsa-miR- | MIMAT0027579 | 3.8915 | 4.91E-288 | 0.8626 | AAGUCCUGCUUC | 90 |
| 6838-3p | | | | | UGUUGCAG | |
| hsa-miR- | MIMAT0014998 | 3.208 | 8.28E-288 | 0.8756 | UAAAGAACUCUU | 91 |
| 3133 | | | | | AAAACCCAAU | |
| hsa-miR- | MIMAT0018114 | 2.7899 | 1.62E-287 | 0.8698 | AUCUGUAAGAGA | 92 |
| 3686 | | | | | AAGUAAAUGA | |
| hsa-miR- | MIMAT0030996 | −0.644 | 2.83E-287 | 0.1334 | GGAUGGUUGGGG | 93 |
| 8069 | | | | | GCGGUCGGCGU | |
| hsa-miR- | MIMAT0017990 | 2.6406 | 9.09E-287 | 0.8664 | UGUUGUACUUUU | 94 |
| 3613-5p | | | | | UUUUUUGUUC | |
| hsa-miR- | MIMAT0019924 | 3.7337 | 1.04E-286 | 0.8685 | UGAGAUGACACU | 95 |
| 4770 | | | | | GUAGCU | |
| hsa-miR- | MIMAT0018095 | 3.2393 | 1.15E-286 | 0.876 | AUGAGACUCAUG | 96 |
| 3672 | | | | | UAAAACAUCUU | |
| hsa-miR- | MIMAT0019968 | 3.0329 | 1.15E-286 | 0.8751 | AGAAGUGGCUAA | 97 |
| 4795-5p | | | | | UAAUAUUGA | |
| hsa-miR- | MIMAT0019810 | 2.7937 | 1.36E-285 | 0.8686 | AGCAAGGCGGCA | 98 |
| 4708-3p | | | | | UCUCUCUGAU | |
| hsa-miR- | MIMAT0017984 | 3.3113 | 1.12E-284 | 0.8716 | GCAUGUGAUGAA | 99 |
| 3607-5p | | | | | GCAAAUCAGU | |
| hsa-miR- | MIMAT0019203 | 3.1541 | 1.27E-284 | 0.8715 | UGGCCCAACCUA | 100 |
| 3136-3p | | | | | UUCAGUUAGU | |
| hsa-miR- | MIMAT0026722 | 3.2543 | 1.60E-284 | 0.8766 | AAGCUUUUUGCU | 101 |
| 208b-5p | | | | | CGAAUUAUGU | |
| hsa-miR- | MIMAT0004694 | 2.0714 | 3.09E-284 | 0.8813 | AGGGGUGCUAUC | 102 |
| 342-5p | | | | | UGUGAUUGA | |
| hsa-miR- | MIMAT0019078 | 3.4958 | 4.06E-284 | 0.8692 | UGUGGUAGAUAU | 103 |
| 4536-5p | | | | | AUGCACGAU | |
| hsa-miR- | MIMAT0018354 | 2.9793 | 4.30E-284 | 0.867 | AAAAGUAAUCAC | 104 |
| 548y | | | | | UGUUUUUGCC | |
| hsa-miR- | MIMAT0019197 | 2.9432 | 1.14E-283 | 0.8699 | AGACACUAUACG | 105 |
| 3117-5p | | | | | AGUCAUAU | |
| hsa-miR- | MIMAT0022291 | 2.8156 | 1.90E-283 | 0.8721 | AAAAGUAAUUGC | 106 |
| 548au-5p | | | | | GGUUUUUGC | |
| hsa-miR- | MIMAT0018988 | 3.0491 | 3.13E-283 | 0.8699 | AAGGUUUGGAUA | 107 |
| 4464 | | | | | GAUGCAAUA | |
| hsa-miR- | MIMAT0004514 | 4.5967 | 5.44E-282 | 0.8495 | GCUGGUUUCAUA | 108 |
| 29b-1-5p | | | | | UGGUGGUUUAGA | |
| hsa-miR- | MIMAT0019025 | 4.2034 | 3.12E-281 | 0.8496 | UCUGGUAAGAGA | 109 |
| 4490 | | | | | UUUGGGCAUA | |
| hsa-miR- | MIMAT0022303 | 3.0952 | 4.00E-281 | 0.8747 | AAAAGUACUUGC | 110 |
| 548av-5p | | | | | GGAUUU | |
| hsa-miR- | MIMAT0018085 | 0.845 | 1.01E-280 | 0.864 | UGAGCACCACAC | 111 |
| 3663-3p | | | | | AGGCCGGGCGC | |
| hsa-miR- | MIMAT0018078 | 3.3518 | 1.62E-280 | 0.8727 | UUUAAGAAAACA | 112 |
| 3658 | | | | | CCAUGGAGAU | |
| hsa-miR- | MIMAT0027670 | 1.0643 | 2.11E-280 | 0.8637 | AGGGGGGCACUG | 113 |
| 6885-5p | | | | | CGCAAGCAAAGC | |
| | | | | | C | |
| hsa-miR- | MIMAT0019914 | 3.0749 | 1.52E-278 | 0.866 | UGGAUGUGGAAG | 114 |
| 4764-5p | | | | | GAGUUAUCU | |
| hsa-miR- | MIMAT0027431 | 1.4747 | 2.17E-278 | 0.852 | UCACCUGGCUGG | 115 |
| 6765-3p | | | | | CCCGCCCAG | |
| hsa-miR- | MIMAT0019903 | 0.7322 | 3.24E-278 | 0.8636 | GUGAGUGGGAGC | 116 |
| 4758-5p | | | | | CGGUGGGGCUG | |
| hsa-miR- | MIMAT0026621 | 3.4016 | 1.33E-277 | 0.8701 | AGAAGGCACUAU | 117 |
| 605-3p | | | | | GAGAUUUAGA | |
| hsa-miR- | MIMAT0019953 | 2.7938 | 1.86E-277 | 0.864 | AGCAGAGGCAGA | 118 |
| 2467-3p | | | | | GAGGCUCAGG | |
| hsa-miR- | MIMAT0018186 | 2.7564 | 3.54E-277 | 0.8633 | UAACGCAUAAUA | 119 |
| 3912-3p | | | | | UGGACAUGU | |
| hsa-miR- | MIMAT0022861 | 2.8425 | 7.60E-277 | 0.8656 | GGUGGAUAUUCC | 120 |
| 376c-5p | | | | | UUCUAUGUU | |
| hsa-miR- | MIMAT0004913 | 3.6397 | 1.40E-276 | 0.8615 | UGCAACUUACCU | 121 |
| 891b | | | | | GAGUCAUUGA | |
| hsa-miR- | MIMAT0005573 | 0.7801 | 3.60E-276 | 0.8642 | UGAGCCCCUGUG | 122 |
| 1225-3p | | | | | CCGCCCCCAG | |
| hsa-miR- | MIMAT0026765 | 3.0426 | 3.89E-276 | 0.8682 | AGCUGUAAUUAG | 123 |
| 1537-5p | | | | | UCAGUUUUCU | |
| hsa-miR- | MIMAT0003289 | 2.9115 | 4.35E-276 | 0.869 | AUGGAGAUAGAU | 124 |
| 620 | | | | | AUAGAAAU | |
| hsa-miR- | MIMAT0004792 | 0.8682 | 8.73E-275 | 0.8703 | AGGGACGGGACG | 125 |
| 92b-5p | | | | | CGGUGCAGUG | |
| hsa-miR- | MIMAT0022728 | 2.888 | 1.48E-274 | 0.8664 | UAAAUUUCACCU | 126 |
| 513c-3p | | | | | UUCUGAGAAGA | |
| hsa-miR- | MIMAT0019801 | 3.0443 | 5.33E-274 | 0.8632 | UAGCAAUACAGU | 127 |
| 4703-5p | | | | | ACAAAUAUAGU | |
| hsa-miR- | MIMAT0022270 | 3.0034 | 7.42E-274 | 0.8612 | UUAGCUUAAGGA | 128 |
| 5579-3p | | | | | GUACCAGAUC | |
| hsa-miR- | MIMAT0014980 | 2.7074 | 7.43E-274 | 0.8644 | UGUGACUGCAUU | 129 |
| 3118 | | | | | AUGAAAAUUCU | |
| hsa-miR- | MIMAT0019929 | 3.7663 | 3.25E-273 | 0.8636 | UCUGGUAUGUAG | 130 |
| 4774-5p | | | | | UAGGUAAUAA | |
| hsa-miR- | MIMAT0027650 | −1.025 | 3.42E-273 | 0.1364 | UGAGGGACCCAG | 131 |
| 6875-5p | | | | | GACAGGAGA | |
| hsa-miR- | MIMAT0017983 | 3.0184 | 4.37E-272 | 0.8679 | UUAGUGAAGGCU | 132 |
| 3606-5p | | | | | AUUUUAAUU | |
| hsa-miR- | MIMAT0022705 | 3.0178 | 5.70E-272 | 0.8639 | AUCAUACAAGGA | 133 |
| 539-3p | | | | | CAAUUUCUUU | |
| hsa-miR- | MIMAT0004602 | −2.925 | 2.20E-271 | 0.1132 | ACAGGUGAGGUU | 134 |
| 125a-3p | | | | | CUUGGGAGCC | |
| hsa-miR- | MIMAT0019014 | 4.1598 | 4.56E-271 | 0.8752 | AGCCAAGUGGAA | 135 |
| 4480 | | | | | GUUACUUUA | |
| hsa-miR- | MIMAT0004809 | 3.6281 | 3.24E-270 | 0.8582 | AUGCUGACAUAU | 136 |
| 628-5p | | | | | UUACUAGAGG | |
| hsa-miR- | MIMAT0027398 | 0.6219 | 4.60E-270 | 0.8632 | UCGGGCCUGGGG | 137 |
| 6749-5p | | | | | UUGGGGGAGC | |
| hsa-miR- | MIMAT0031178 | 1.7575 | 5.25E-270 | 0.8649 | AUCCUAGUCACG | 138 |
| 7975 | | | | | GCACCA | |
| hsa-miR- | MIMAT0017988 | 2.885 | 6.46E-270 | 0.8644 | UUGUGAAGAAAG | 139 |
| 3611 | | | | | AAAUUCUUA | |
| hsa-miR- | MIMAT0019901 | 4.1397 | 9.46E-270 | 0.8504 | AGGCCUCUGUGA | 140 |
| 4757-5p | | | | | CGUCACGGUGU | |
| hsa-miR- | MIMAT0004597 | 4.0093 | 2.33E-269 | 0.8405 | UACCACAGGGUA | 141 |
| 140-3p | | | | | GAACCACGG | |
| hsa-miR- | MIMAT0004508 | −1.4 | 2.81E-269 | 0.1278 | GGGUGGGGAUUU | 142 |
| 92a-2-5p | | | | | GUUGCAUUAC | |
| hsa-miR- | MIMAT0019907 | 2.9356 | 9.74E-269 | 0.8569 | AAAUUCAUGUUC | 143 |
| 4760-3p | | | | | AAUCUAAACC | |
| hsa-miR- | MIMAT0019832 | 3.4867 | 3.06E-268 | 0.8605 | UCACAAAUCUAU | 144 |
| 4719 | | | | | AAUAUGCAGG | |
| hsa-miR- | MIMAT0018091 | 3.1826 | 5.97E-268 | 0.8594 | AAUGUAGAGAUU | 145 |
| 3668 | | | | | GAUCAAAAU | |
| hsa-miR- | MIMAT0018094 | 3.1115 | 3.88E-267 | 0.8665 | AUCAAAUAAGGA | 146 |
| 3671 | | | | | CUAGUCUGCA | |
| hsa-miR- | MIMAT0019005 | 2.9265 | 4.21E-267 | 0.8685 | AUUAAGGACAUU | 147 |
| 4477b | | | | | UGUGAUUGAU | |
| hsa-miR- | MIMAT0004698 | 3.4995 | 5.72E-267 | 0.8615 | AUGUAGGGCUAA | 148 |
| 135b-3p | | | | | AAGCCAUGGG | |
| hsa-miR- | MIMAT0025851 | 3.0746 | 6.95E-267 | 0.8526 | CGCGCCUGCAGG | 149 |
| 6720-3p | | | | | AACUGGUAGA | |
| hsa-miR- | MIMAT0022497 | 4.0102 | 1.35E-266 | 0.843 | AAUAAUAUCACA | 150 |
| 5692b | | | | | GUAGGUGU | |
| hsa-miR- | MIMAT0005909 | 2.9262 | 2.18E-266 | 0.8684 | AGUUAGGAUUAG | 151 |
| 1258 | | | | | GUCGUGGAA | |
| hsa-miR- | MIMAT0022269 | 3.0487 | 2.47E-266 | 0.8615 | UAUGGUACUCCU | 152 |
| 5579-5p | | | | | UAAGCUAAC | |
| hsa-miR- | MIMAT0017985 | 3.0664 | 2.78E-266 | 0.87 | ACUGUAAACGCU | 153 |
| 3607-3p | | | | | UUCUGAUG | |
| hsa-miR- | MIMAT0018097 | 2.9167 | 3.13E-266 | 0.8563 | AUUGUAGAACCU | 154 |
| 3674 | | | | | AAGAUUGGCC | |
| hsa-miR- | MIMAT0020959 | 2.9715 | 3.87E-265 | 0.8609 | UCGUGCAUAUAU | 155 |
| 4536-3p | | | | | CUACCACAU | |
| hsa-miR- | MIMAT0019935 | 4.016 | 5.08E-265 | 0.8453 | AUACCUCAUCUA | 156 |
| 4777-3p | | | | | GAAUGCUGUA | |
| hsa-miR- | MIMAT0019779 | 1.1813 | 6.16E-265 | 0.8831 | GAGCAGGCGAGG | 157 |
| 4690-5p | | | | | CUGGGCUGAA | |
| hsa-miR- | MIMAT0010214 | 4.1969 | 9.21E-265 | 0.8436 | UCGAGGAGCUCA | 158 |
| 151b | | | | | CAGUCU | |
| hsa-miR- | MIMAT0022470 | 2.6508 | 1.20E-264 | 0.854 | GUAGCACCUUGC | 159 |
| 5682 | | | | | AGGAUAAGGU | |
| hsa-miR- | MIMAT0018093 | 3.4426 | 4.50E-264 | 0.8522 | AGAGCUCACAGC | 160 |
| 3670 | | | | | UGUCCUUCUCUA | |
| hsa-miR- | MIMAT0019944 | 2.6812 | 2.32E-263 | 0.8577 | UUCUGGAUAUGA | 161 |
| 4782-5p | | | | | AGACAAUCAA | |
| hsa-miR- | MIMAT0003253 | 3.7752 | 2.38E-263 | 0.8486 | UUUCCAUAGGUG | 162 |
| 587 | | | | | AUGAGUCAC | |
| hsa-miR- | MIMAT0018944 | 1.6684 | 5.66E-263 | 0.8711 | AAAAGCUGGGCU | 163 |
| 4429 | | | | | GAGAGGCG | |
| hsa-miR- | MIMAT0014983 | 3.2803 | 3.74E-261 | 0.8688 | UAAAUAGAGUAG | 164 |
| 3121-3p | | | | | GCAAAGGACA | |
| hsa-miR- | MIMAT0019711 | 1.1274 | 5.05E-261 | 0.8517 | UGGGCGAGGGGU | 165 |
| 4649-5p | | | | | GGGCUCUCAGAG | |
| hsa-miR- | MIMAT0018199 | 2.8275 | 5.44E-261 | 0.8637 | AUAUGUAUAUGU | 166 |
| 3924 | | | | | GACUGCUACU | |
| hsa-miR- | MIMAT0005945 | 3.0436 | 1.03E-260 | 0.8613 | CGGAUGAGCAAA | 167 |
| 1255b-5p | | | | | GAAAGUGGUU | |
| hsa-miR- | MIMAT0019807 | 0.5556 | 1.92E-260 | 0.8618 | GCCCCGGCGCGG | 168 |
| 4707-5p | | | | | GCGGGUUCUGG | |
| hsa-miR- | MIMAT0000422 | 3.6633 | 7.51E-260 | 0.8605 | UAAGGCACGCGG | 169 |
| 124-3p | | | | | UGAAUGCCAA | |
| hsa-miR- | MIMAT0031003 | 2.8287 | 1.07E-259 | 0.8548 | UAUAUGGACUUU | 170 |
| 8076 | | | | | UCUGAUACAAUG | |
| hsa-miR- | MIMAT0019816 | 3.0683 | 1.30E-259 | 0.8602 | UGCAUCAGGCCA | 171 |
| 4711-5p | | | | | GAAGACAUGAG | |
| hsa-miR- | MIMAT0004699 | 3.4387 | 1.84E-259 | 0.8507 | AAGUUCUGUUAU | 172 |
| 148b-5p | | | | | ACACUCAGGC | |
| hsa-miR- | MIMAT0000101 | 4.2517 | 2.98E-259 | 0.838 | AGCAGCAUUGUA | 173 |
| 103a-3p | | | | | CAGGGCUAUGA | |
| hsa-miR- | MIMAT0018202 | 2.9326 | 2.03E-258 | 0.8568 | CAGGUAGAUAUU | 174 |
| 3927-3p | | | | | UGAUAGGCAU | |
| hsa-miR- | MIMAT0023713 | 0.8116 | 2.43E-258 | 0.8457 | AGAGAUGAAGCG | 175 |
| 6088 | | | | | GGGGGGCG | |
| hsa-miR- | MIMAT0035704 | 3.2782 | 3.45E-258 | 0.856 | CAAAAACCAUAG | 176 |
| 548bb-3p | | | | | UUACUUUUGC | |
| hsa-miR- | MIMAT0019218 | 3.5776 | 3.49E-258 | 0.8605 | AGCUCUGCUGCU | 177 |
| 3194-3p | | | | | CACUGGCAGU | |
| hsa-miR- | MIMAT0022500 | 3.6423 | 4.71E-258 | 0.8542 | UUCUGGAUAACA | 178 |
| 5706 | | | | | UGCUGAAGCU | |
| hsa-miR- | MIMAT0005935 | 2.5997 | 5.30E-258 | 0.8516 | AAAAGUAAUUGC | 179 |
| 548i | | | | | GGAUUUUGCC | |
| hsa-miR- | MIMAT0022285 | 3.3468 | 1.65E-257 | 0.8526 | UGAAGUACCAGC | 180 |
| 5585-5p | | | | | UACUCGAGAG | |
| hsa-miR- | MIMAT0005948 | 3.025 | 2.09E-257 | 0.8568 | ACUGGCUAGGGA | 181 |
| 664a-5p | | | | | AAAUGAUUGGAU | |
| hsa-miR- | MIMAT0005931 | 3.0658 | 2.19E-257 | 0.8615 | UAAGUGCUUCCA | 182 |
| 302e | | | | | UGCUU | |
| hsa-miR- | MIMAT0019016 | 2.8533 | 5.07E-257 | 0.8482 | AACCCAGUGGGC | 183 |
| 4482-5p | | | | | UAUGGAAAUG | |
| hsa-miR- | MIMAT0030988 | 2.8829 | 9.78E-257 | 0.8618 | CUUAGAUUAGAG | 184 |
| 8061 | | | | | GAUAUUGUU | |
| hsa-miR- | MIMAT0003220 | 3.3066 | 1.57E-256 | 0.8556 | GAUGAGCUCAUU | 185 |
| 556-5p | | | | | GUAAUAUGAG | |
| hsa-miR- | MIMAT0018182 | 2.8977 | 2.65E-256 | 0.8544 | GAGCAAUGUAGG | 186 |
| 3908 | | | | | UAGACUGUUU | |
| hsa-miR- | MIMAT0000418 | 3.3977 | 3.62E-255 | 0.8559 | AUCACAUUGCCA | 187 |
| 23b-3p | | | | | GGGAUUACCAC | |
| hsa-miR- | MIMAT0027404 | −0.394 | 1.30E-254 | 0.145 | GGGGGGUGUGGA | 188 |
| 6752-5p | | | | | GCCAGGGGGC | |
| hsa-miR- | MIMAT0019848 | 2.4885 | 1.43E-254 | 0.8753 | AUAGUGGGAAGC | 189 |
| 4727-3p | | | | | UGGCAGAUUC | |
| hsa-miR- | MIMAT0004917 | 3.3784 | 2.36E-254 | 0.8465 | GACUGACACCUC | 190 |
| 888-3p | | | | | UUUGGGUGAA | |
| hsa-miR- | MIMAT0018946 | 2.8037 | 3.21E-254 | 0.8472 | GAAAACGACAAU | 191 |
| 548ad-3p | | | | | GACUUUUGCA | |
| hsa-miR- | MIMAT0003258 | 3.3229 | 7.95E-254 | 0.8404 | GAGCUUAUUCAU | 192 |
| 590-5p | | | | | AAAAGUGCAG | |
| hsa-miR- | MIMAT0019925 | 3.8234 | 2.08E-253 | 0.8633 | AGCAGACUUGAC | 193 |
| 4771 | | | | | CUACAAUUA | |
| hsa-miR- | MIMAT0026624 | 2.7692 | 2.57E-253 | 0.8523 | AAAGGAAAGUGU | 194 |
| 651-3p | | | | | AUCCUAAAAG | |
| hsa-miR- | MIMAT0018979 | 3.5026 | 5.50E-252 | 0.8513 | UCACAAGGUAUU | 195 |
| 4457 | | | | | GACUGGCGUA | |
| hsa-miR- | MIMAT0019745 | 3.1318 | 2.17E-251 | 0.8469 | AGGGAAAAAAAA | 196 |
| 4668-5p | | | | | AAGGAUUUGUC | |
| hsa-miR- | MIMAT0005901 | 0.7246 | 3.23E-251 | 0.8513 | ACGCCCUUCCCC | 197 |
| 1249-3p | | | | | CCCUUCUUCA | |
| hsa-miR- | MIMAT0005896 | 2.7578 | 3.23E-251 | 0.8542 | AAGUAGUUGGUU | 198 |
| 1244 | | | | | UGUAUGAGAUGG | |
| | | | | | UU | |
| hsa-miR- | MIMAT0022712 | 2.5977 | 4.33E-251 | 0.8494 | AGUGCCUGCUAU | 199 |
| 1271-3p | | | | | GUGCCAGGCA | |
| hsa-miR- | MIMAT0014991 | 2.6666 | 2.22E-250 | 0.8493 | UCUGGCAAGUAA | 200 |
| 3128 | | | | | AAAACUCUCAU | |

Note:
miRNA differential expression analysis comparing 1408 cancer patients across 7 cancer types and 1408 matched non-cancer controls in the Train Set was performed by limma. miRNAs were ranked by adjusted p values (calculated by limma), and top 200 miRNAs were derived from the ranked list.

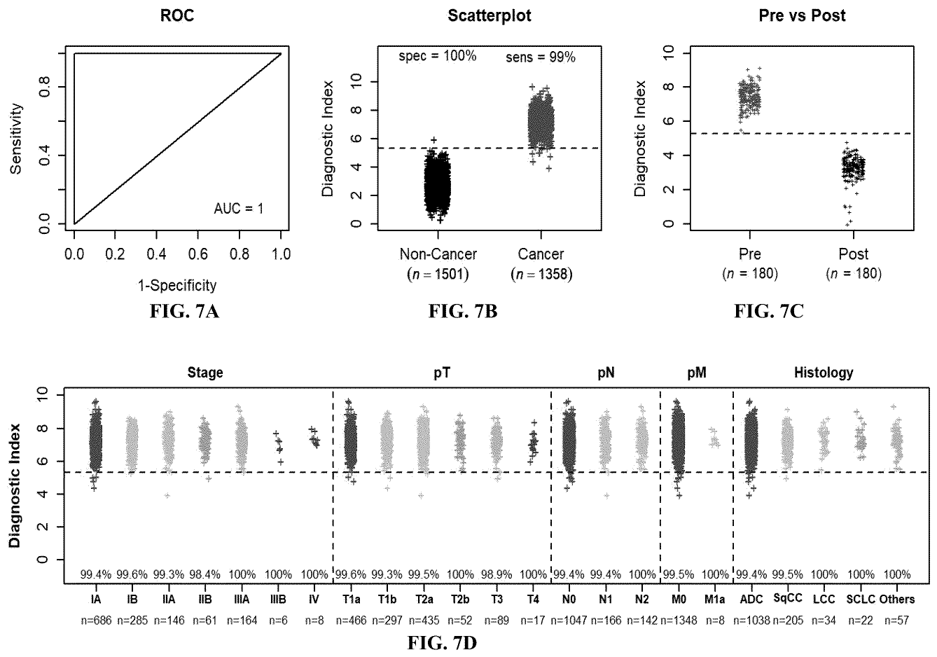

Validation of the Diagnostic Model in the Independent Validation Set 1: The performance of the 4-miRNA model was first evaluated in the independent Validation Set 1 (n=2859) that included 1358 lung cancer patients and 1501 non-cancer controls. The model achieved an AUC of 1.000 (FIG. 7A) with a specificity of 100% and sensitivity of 99% (FIG. 7B). In addition, analysis of paired serum samples (pre- vs. post-surgery; n=180) verified normalization of the diagnostic indices to the levels of non-cancer controls in post-surgery serum samples (FIG. 7C). Furthermore, the performance of the 4-miRNA model was evaluated across clinical subsets of the Validation Set 1, as defined by the clinical stages, TNM stages, and histology subtypes. High sensitivities were observed for all clinical subsets. The model achieved at least 99% sensitivity for 22 out of 24 clinical subsets examined except for stage IIB and T3 tumors (FIG. 7D). In particular, the model demonstrated >99% sensitivities for stage I lung cancers and for adenocarcinoma and squamous cell carcinoma.

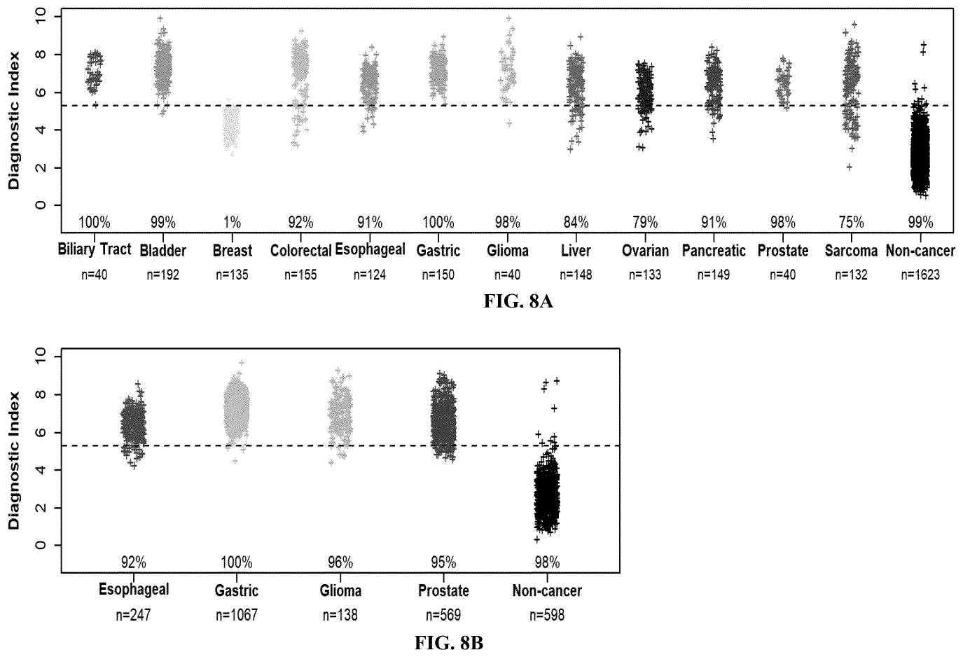

Validation of the Diagnostic Model in the Independent Validation Sets 2 and 3: The independent Validation Set 2 included 1438 patients across 12 additional cancer types and 1623 non-cancer controls. Except for breast cancer, the 4-miRNA model achieved at least 90% sensitivity for eight cancer types (biliary tract, bladder, colorectal, esophageal, gastric, glioma, pancreatic and prostate) and at least 75% for the other three cancer types (liver, ovarian and sarcoma) (FIG. 8A and Table 2). Noteworthy, while the model had a reasonable AUC value of 0.909 for breast cancer, the 1% sensitivity was still very low due to the high specificity requirement (FIG. 8A and Table 2). The independent Validation Set 3 included 2079 patients from four cancer types (esophageal, gastric, glioma and prostate) and 598 non-cancer controls, where the sample sizes of the four cancer types were substantially larger than those in Validation Set 2 (247 vs. 124 for esophageal, 1067 vs. 150 for gastric, 196 vs. 40 for glioma, and 569 vs. 40 for prostate). The 4-miRNA model achieved >0.99 AUC and >99% sensitivity for all four cancer types, similar to those observed in Validation Set 2 (FIG. 8B and Table 2). The specificity of the model was a little lower in Validation Set 3 than in Validation Set 2 (0.98 vs 0.99) (FIG. 8B). Therefore, for Validation Set 3, a sensitivity analysis with an adjusted diagnostic index cut-point of 5.6 was explored to increase the specificity of the new model to 99%. With this new cut-point, the model still achieved high sensitivity for all four cancer types, including 99% for gastric, 92% for glioma, 91% for prostate, and 89% for esophageal cancers.

### Discussion

Noninvasive screening tests for MCED via analyzing circulating cell-free nucleic acids and/or proteins in the body fluid, especially blood, have attracted high attention for the last decade. In this study, we reported the development and validation of a serum 4-miRNA diagnostic model and demonstrated that in three large independent validation sets totaling 8597 participants (4875 cancer patients across 13 cancer types and 3722 non-cancer individuals), the 4-miRNA model can detect 12 cancer types simultaneously with high sensitivities (>90% for 9 cancer types, and >75% for 3 cancer types) while still achieving a very high specificity of ~99%. In addition, the observation that the diagnostic indices for the post-surgery serum samples were reduced to normal levels suggests the potential utility of the model for monitoring response to treatment and detection of recurrence.

Importantly, our model was able to detect early-stage cancers at high sensitivity. Specifically, in Validation Set 1 of lung cancer patients, the model detects stage I and II cancers at a sensitivity ranging from 98.4% to 99.6% (FIG. 7D). In Validation Sets 2 and 3, while individual patient-level stage information was not available, aggregate stage information was provided for 6 of the 12 cancer types examined. First, all gastric cancer patients were stage I or II, thus the 100% sensitivity of our model applied to early-stage gastric cancer. Second, 88% and 93% of bladder and prostate cancer patients had node negative disease. Thus, with 99% and 98% sensitivity for these two cancers, the sensitivity for stage I or II bladder and prostate cancers should be very high as well. Third, 66% and 70% of esophageal and liver cancer patients were stage I or II, respectively. It was reasonable to speculate that the sensitivity for stage I or II of these two cancers should not be far off from the 92% and 84% sensitivity reported for all stages included. In summary, based on the data currently available in the three validation sets, we concluded that our 4-miRNA model achieves high sensitivity for stage I or II disease of six cancer types (lung, gastric, bladder, prostate, esophageal, and liver).

It is worth noting that a simple four-parameter diagnostic model like the one described here not only costs significantly less, but also can be developed into an in vitro diagnostic (IVD) test using RT-qPCR capable of decentralized testing, which has an advantage over NGS-based tests that are usually implemented as a laboratory developed test (LDT). These characteristics are important to drive adoption and increase affordability of MCED tests as they are intended to target high risk or at-risk general public, especially for those from low-income communities.

In summary, our study has provided proof-of-concept data for developing a blood screening test based on expression profiles of circulating cell-free miRNAs for 12 cancer types, which account for 50% estimated new cancer cases and 63% cancer deaths in the US in 2022 (Siegel R L, et al., 2022).

### REFERENCES

Zhang C, et al. Analysis of differential gene expression and novel transcript units of ovine muscle transcriptomes. PLoS One. 2014 Feb. 26; 9(2):e89817.
Ritchie, M E; et al. (2015). limma powers differential expression analyses for RNA-sequencing and microarray studies. Nucleic Acids Research 43(7), e47.
Venables, W N and Ripley, BD (2002) Modern Applied Statistics with S. Fourth edition. Springer.
Tibshirani, Robert (1996). “Regression Shrinkage and Selection via the lasso”. Journal of the Royal Statistical Society. Series B (methodological). Wiley. 58 (1): 267-88.
Hoerl, Arthur E.; Kennard, Robert W. (1970). “Ridge Regression: Biased Estimation for Nonorthogonal problems”. Technometrics. 12 (1): 55-67.
Ripley, B. D. (1996) Pattern Recognition and Neural Networks. Cambridge University Press.
Zhang A, et al. A Novel Blood-Based microRNA Diagnostic Model with High Accuracy for Multi-Cancer Early Detection. Cancers 2022, 14(6), 1450.
Zhang A and Hu H. Independent validation of a novel noninvasive 4-microRNA diagnostic model for multicancer early detection. Journal of Clinical Oncology. 2022; 40(16_suppl):3065-3065.
Asakura K, et al. A miRNA-based diagnostic model predicts resectable lung cancer in humans with high accuracy. Commun Biol. 2020; 3(1).
Yokoi A, et al. Integrated extracellular microRNA profiling for ovarian cancer screening. Nat Commun. 2018; 9(1).
Yamamoto Y, et al. Highly Sensitive Circulating MicroRNA Panel for Accurate Detection of Hepatocellular Carcinoma in Patients With Liver Disease. Hepatol Commun. 2020; 4(2):284-297.
Usuba W, et al. Circulating miRNA panels for specific and early detection in bladder cancer. Cancer Sci. 2019; 110(1).
Sudo K, et al. Development and Validation of an Esophageal Squamous Cell Carcinoma Detection Model by Large-Scale MicroRNA Profiling. JAMA Netw Open. 2019; 2(5):e194573.
Abe S, et al. A novel combination of serum microRNAs for the detection of early gastric cancer. Gastric Cancer. 2021; 24(4):835-843.
Ohno M, et al. Assessment of the Diagnostic Utility of Serum MicroRNA Classification in Patients With Diffuse Glioma. JAMA Netw Open. 2019; 2(12):e1916953.
Urabe F, et al. Large-scale Circulating microRNA Profiling for the Liquid Biopsy of Prostate Cancer. Clinical Cancer Research. 2019; 25(10):3016-3025.
Siegel R L, et al. Cancer statistics, 2022. CA Cancer J Clin. 2022; 72(1):7-33.

1. A method of developing a diagnostic model for detecting a cancer of interest, comprising the steps of:
(1) constructing a training set that comprises expression profiles of a plurality of miRNAs obtained from non-cancer subjects and cancer patients, wherein there are at least two cancer types among the cancer patients; and
(2) developing the diagnostic model based on the training set, comprising the substeps of:
(a) performing differential expression analysis over the expression profiles of the plurality of miRNAs by means of a first statistical model such that the plurality of miRNAs are ranked by adjusted p values; and
(b) building the diagnostic model based on a selected miRNA biomarker set from the plurality of miRNAs, wherein the selected miRNA biomarker set comprises at least one miRNA, each having a rank no greater than a preset cutoff m, where m is an integer greater than zero.

2. The method of claim 1, wherein the substep (b) in step (2) comprises:
calculating a diagnostic index based on the expression profile of the selected miRNA biomarker set, wherein the diagnostic index is calculated based on formula:
$$diagnostic index = {\sum }_{i = 1}^{n} {t}_{i} * {miRNA}_{i} ; \qquad ( I )$$
where n is the total number of the at least one miRNA in the selected miRNA biomarker set, n is an integer no greater than m, miRNA, is the expression level of i^{th} miRNA in the selected miRNA biomarker set, i is an integer greater than zero and smaller than or equal to n, and tᵢ is a weight for the i^{th} miRNA.

3. The method of claim 2, wherein tᵢ is a constant number and/or tᵢ is based on a second statistical model, selected from one of linear model for microarray data (limma) model, logistic regression model, linear discriminant analysis (LDA) model, conditional logistic regression model, lasso regression model, ridge regression model, random forest, support vector machine, or probit regression model.

4. (canceled)

5. The method of claim 4, wherein the first statistical model and the second statistical model are substantially the same.

6. The method of claim 5, wherein limma model is used as both the first statistical model and the second statistical model.

7. The method of claim 2, wherein the at least one miRNA in the selected miRNA biomarker set are respectively top n ranked miRNAs.

8. The method of claim 1, wherein step (2) further comprises the substep of:
(c) evaluating performance of the diagnostic model through cross validation in the training set.

9. (canceled)

10. (canceled)

11. The method of claim 1, further comprising:
(3) validating the diagnostic model based on a validation set, wherein the validation set comprises expression profiles of the selected miRNA biomarker set obtained from non-cancer subjects and cancer patients with the cancer of interest.

12. The method of claim 11, wherein the step (3) comprises at least one of:
calculating AUC of ROC curves, and evaluating the performance of the diagnostic model based thereon; or
calculating specificity and sensitivity of the diagnostic model, and evaluating the performance of the diagnostic model based thereon.

13. (canceled)

14. (canceled)

15. A method for detecting a cancer of interest from a subject by means of a diagnostic model, developed by the method according to claim 1, comprising:
determining an expression profile of the selected miRNA biomarker set from a biological sample obtained from the subject;
calculating a diagnostic index of the biological sample based on the expression profile of the selected miRNA biomarker set, wherein the diagnostic index is calculated based on formula:
$$diagnostic index = {\sum }_{i = 1}^{n} {t}_{i} * {miRNA}_{i} ; \qquad ( I )$$
where n is the total number of the at least one miRNA in the selected miRNA biomarker set, miRNAᵢ is the expression level of i^{th} miRNA in the selected miRNA biomarker set, i is an integer greater than zero and smaller than or equal to n; and tᵢ is a weight for the i^{th} miRNA; and
classifying the subject as having the cancer of interest or not based on the calculated diagnostic index, wherein the subject is classified as having the cancer of interest if the calculated diagnostic index is greater than or equal to a pre-determined threshold or as not having the cancer of interest if otherwise.

16. (canceled)

17. The method of claim 15, wherein each miRNA in the selected miRNA biomarker set is from the top 200 miRNAs as listed in TABLE 1, and n is smaller than or equal to 200.

18. The method of claim 15, wherein the diagnostic index is calculated via a weighted model using weights from the limma model.

19. The method of claim 18, wherein the at least one miRNA in the selected miRNA biomarker set are respectively top n ranked miRNAs.

20. The method of claim 19, wherein n is no less than 4 and no more than 200, wherein the classification is capable of achieving an AUC of more than approximately 0.97 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer.

21. The method of claim 19, wherein n is no less than 4 and no more than 200, wherein the classification is capable of achieving a sensitivity of at least approximately 0.75 while maintaining a specificity of no less than approximately 0.99 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer.

22. (canceled)

23. (canceled)

24. (canceled)

25. (canceled)

26. The method of claim 15, wherein the selected miRNA biomarker set consists of hsa-miR-5100, hsa-miR-1228-5p, hsa-miR-8073, and hsa-miR-663a.

27. The method of claim 26, wherein the classification is capable of achieving a sensitivity of at least approximately 0.75 while maintaining a specificity of no less than approximately 0.99 for detecting lung cancer, colorectal cancer, esophageal cancer, gastric cancer, liver cancer, ovarian cancer, pancreatic cancer, sarcoma, biliary tract cancer, bladder cancer, glioma, or prostate cancer.

28. (canceled)

29. (canceled)

30. (canceled)

31. (canceled)

32. (canceled)

33. (canceled)

34. (canceled)

35. (canceled)

36. The method of claim 15, wherein the biological sample is a liquid biopsy sample selected from a group consisting of a blood sample, a serum sample, a plasma sample, a urine sample, a saliva sample, and a sputum sample.

37. A system for detecting a cancer of interest from a subject, comprising:
a processor; and
a non-transitory storage medium containing program instructions for execution by the processor, wherein the program instructions cause the processor to execute steps in the method according to claim 15.

38. A non-transitory storage medium, storing computer-executable program instructions which, when executed by a processor, cause the processor to execute the method according to claim 15.
